(19) United States
(12) Patent Application Publication
(10) Pub. No.: US 2022/0002785 A1
(43) Pub. Date: Jan. 6, 2022

(54) RAPID IDENTIFICATION OF BACTERIAL PATHOGENS

(71) Applicant: MASSEY UNIVERSITY, Palmerston North (NZ)

(72) Inventors: Peter LOCKHART, Palmerston North (NZ); Richard WINKWORTH, Palmerston North (NZ); Patricia MCLENACHAN, Palmerston North (NZ); Nicole GRÜNHEIT, Dußlingen (DE); Richard FONG, Palmerston North (NZ)

(21) Appl. No.: 17/292,341

(22) PCT Filed: Nov. 8, 2019
(86) PCT No.: PCT/IB2019/059590

§ 371 Date: May 7, 2021

(30) Foreign Application Priority Data
Nov. 9, 2018 (AU) 2018904268

(51) Int. Cl. C12Q 1/689

(52) U.S. Cl. CPC C12Q 1/689; C12Q 2600/156; C12Q 2600/106

(57) ABSTRACT

Disclosed herein are methods and compositions for specific detection of Mycobacterium spp. in a sample and for profiling multiple gene loci within Mycobacterium spp. that are linked to or that are directly involved in antibiotic resistance. In particular the method employs a unique set of nucleic acid amplification primers that enable the whole genome sequence-based approach disclosed herein, allowing for the full characterization of the antibiotic resistance profile of Mycobacterium spp.

### FIELD OF THE INVENTION

This invention generally relates to methods and compositions for detecting Mycobacterium tuberculosis in a sample and for profiling multiple gene loci within M. tuberculosis that are linked to or that are directly involved in antibiotic resistance in M. tuberculosis.

### BACKGROUND

Tuberculosis (TB) is an infectious disease usually caused by members of the Mycobacterium tuberculosis species complex. This complex includes four species—M. tuberculosis, M. bovis, M. africanum, and M. microti. These species differ markedly in their epidemiology yet are genetically very similar, 85-100% identity at the DNA level.

In humans TB is curable, yet the disease kills 1.8 million people annually (WHO Global Tuberculosis Report 2016). Additionally, 1.7 billion people have latent TB. Typically, these individuals are asymptomatic but may develop the disease if they become immunocompromised (Houben & Dodd 2016). Despite significant financial input current methods of diagnosing TB have failed to reduce rates of infection and this is impeding current efforts by the World Health Organization (WHO) to eradicate TB (Klopper et al. 2013; Callaway 2017).

Bovine TB (bTB) is an infectious disease caused by M. bovis. This chronic disease affects cattle, other domestic and wild animals, and may also cause disease in humans. Globally, bTB is recognised as one of the seven most neglected endemic zoonoses. Where it occurs, the disease has important socio-economic and public health-related impacts and is a serious constraint on the trade of animals and their products.

The emergence of multi-drug resistant (MDR) and extensively-drug resistant (XDR) strains of TB is a significant obstacle to the control of TB (Nguyen 2017). The former are resistant to one or more first-line drugs such as isoniazid (INH) and rifampicin (RIF) with the latter also resistant to fluoroquinolone (FLQ) and one or more of the second-line drugs such as capreomycin (CPR), kanamycin (KAN), ofloxacin (OFX) and amikacin (AMK) (Migliori et al. 2008). Of perhaps greatest concern is the recent emergence of what appear to be totally-drug resistant strains in South Africa (Klopper et al. 2013).

Used for decades, conventional light microscopy followed by culturing remains the “gold-standard” for diagnosis of active pulmonary TB. Identification of Ziehl-Neelsen stained M. tuberculosis under a light microscope is both rapid and inexpensive. However, once identified the bacterium must be grown in culture for 4-8 weeks before phenotypic drug-susceptibility testing, which can take a further 6 weeks, is conducted. Only at the end of this process can the resistance profile and, therefore, the drug susceptibility of the strain be determined. A patient could, therefore, wait up to 14 weeks to receive appropriate treatment.

More recently, molecular approaches to evaluating resistance profiles have been developed. Commercially available Hain line probe assays utilise a combination of PCR and DNA-DNA hybridization to simultaneously identify M. tuberculosis and detect mutations associated with resistance to several antibiotics (Dookie et al. 2018). The open-tube format of line probe assays is a disadvantage and requires specialised infrastructure. In 2010, the WHO endorsed the use of the cartridge-based Xpert MTB/RIF test for detection of M. tuberculosis. Although the Xpert MTB/RIF was heralded as a significant breakthrough, TB rates have not fallen dramatically since its introduction (Callaway 2017). This may reflect limitations of the Xpert MTB/RIF test. These include the high cost of the device and test cartridges as well as the limited shelf life of test cartridges. Perhaps more importantly, the Xpert MTB/RIF also requires an air-conditioned facility with constant electrical supply and must be regularly maintained (Kane et al. 2016; Evans 2011). In countries where TB is prevalent these latter requirements are often difficult to meet in anything other than a central facility. That the Xpert MTB/RIF cannot be deployed to low-resource settings limits its usefulness.

A limitation common to all existing approaches to the molecular diagnosis of drug-resistant M. tuberculosis is their inability to fully characterise the antibiotic resistance profile of any given strain of M. tuberculosis.

An alternative to DNA amplification-based testing involves the analysis of data from Whole Genome Sequencing (WGS). This approach allows genome wide assessment of genetic mutation, using WGS it is possible to recognise known resistance-inducing sequence variants and to identify novel ones. As a result, WGS has become the first choice for TB diagnosis in research laboratory settings, especially when MDR-TB and XDR-TB are expected (Gilpin et al. 2016). The increased affordability and speed of DNA sequencing as well as the emergence of personal DNA sequencing devices (e.g., Oxford Nanopore MinION) make WGS an increasingly attractive option for rapid TB diagnosis. However, obstacles remain. For example, WGS analyses of sputum DNA from patients may not contain sufficient sequence reads from M. tuberculosis to identify antibiotic resistance inducing mutations and determine, with confidence, an antibiotic resistance profile (Doherty 2014; Brown et al. 2015).

Accordingly, there is a need in the art for alternative methods of detecting M. tuberculosis and M. bovis, and of profiling the antibiotic resistance of various strains of M. tuberculosis and M. bovis that can be carried out more rapidly, at a reduced cost, and in low infrastructure situations.

It is an object of the present invention to go at least some way towards addressing this need, and/or to provide a rapid, low-cost approach for selective amplification of M. tuberculosis DNA using Multiple Displacement Amplification (MDA), and/or to provide a method and composition for detecting M. tuberculosis and for profiling multiple gene loci that are linked to or that are directly involved in antibiotic resistance in M. tuberculosis, and/or to provide a method and composition for detecting M. bovis and for profiling multiple gene loci that are linked to or that are directly involved in antibiotic resistance in M. bovis and/or to at least provide the public with a useful choice.

### SUMMARY OF THE INVENTION

In one aspect the invention relates to a composition comprising 7 to 12 unique oligonucleotide primers, each primer consisting of 11 or 12 nucleotides, wherein each of these oligonucleotide primers specifically binds to a nucleic acid sequence in the M. tuberculosis genome. In one embodiment the composition comprises 7 to 15 unique oligonucleotide primers.

In another aspect the present invention relates to a composition comprising at least 7 unique oligonucleotide primers selected from the group consisting of P1 (SEQ ID NO: 1), P2 (SEQ ID NO: 2), P3 (SEQ ID NO: 3), P4 (SEQ ID NO: 4), P5 (SEQ ID NO: 5), P6 (SEQ ID NO: 6), P7 (SEQ ID NO: 7), P8 (SEQ ID NO: 8), P9 (SEQ ID NO: 9), P10 (SEQ ID NO: 10), P11 (SEQ ID NO: 11), P12 (SEQ ID NO: 12), P13 (SEQ ID NO: 13), P14 (SEQ ID NO: 14) and P15 (SEQ ID NO: 15).

In another aspect the invention relates to a kit comprising at least 7 unique oligonucleotide primers selected from the group consisting of P1-P14 and P15, and at least one enzyme that catalyzes nucleic acid replication.

In another aspect the invention relates to a method of selectively amplifying the genomic DNA of at least one bacterial species or strain from a sample, the method comprising:
contacting the sample with a composition comprising 7-12 unique oligonucleotide primers, each primer consisting of 11 or 12 nucleotides, wherein each of these oligonucleotide primers specifically binds to a nucleic acid sequence in the genome of the bacterial species or strain,
selectively amplifying DNA from the bacterial species or strain of interest in a multiple displacement amplification (MDA) reaction,
identifying from among the selectively amplified DNA, DNA sequences that are assigned with high confidence to the genome of the bacterial species or strain of interest.

In another aspect the invention relates to a method of selectively amplifying the genomic DNA of Mycobacterium tuberculosis from a sample, the method comprising:
contacting the sample with a composition comprising 7 to 12 unique oligonucleotide primers selected from the group consisting of P1-P14 and P15,
selectively amplifying DNA from M. tuberculosis in a multiple displacement amplification (MDA) reaction, and
identifying from among the selectively amplified DNA, DNA sequences that are assigned with high confidence to the genome of M. tuberculosis.

In another aspect the invention relates to a method of selectively amplifying the genomic DNA of Mycobacterium bovis from a sample, the method comprising:
contacting the sample with a composition comprising 7 to 12 unique oligonucleotide primers selected from the group consisting of P1-P14 and P15,
selectively amplifying DNA from M. bovis in a multiple displacement amplification (MDA) reaction, and
identifying from among the selectively amplified DNA, DNA sequences that are assigned with high confidence to the genome of M. bovis.

In another aspect the invention relates to a method of determining the antibiotic resistance profile of a strain of M. tuberculosis, the method comprising:
contacting a sample containing or suspected of containing M. tuberculosis with a composition comprising 7 to 12 unique oligonucleotide primers selected from the group consisting of P1-P14 and P15,
selectively amplifying DNA from Mycobacterium tuberculosis in a multiple displacement amplification (MDA) reaction, and
identifying within the pool of selectively amplified DNA, DNA sequences that encode M. tuberculosis gene products that are linked to, or that are directly involved in, antibiotic resistance in M. tuberculosis.

In another aspect the invention relates to a method of determining the antibiotic resistance profile of a strain of M. bovis, the method comprising:
contacting a sample containing or suspected of containing M. tuberculosis with a composition comprising 7 to 12 unique oligonucleotide primers selected from the group consisting of P1-14 and P15,
selectively amplifying DNA from M. bovis in a multiple displacement amplification (MDA) reaction, and
identifying within the pool of selectively amplified DNA, DNA sequences that encode M. bovis gene products that are linked to, or that are directly involved in, antibiotic resistance in M. bovis.

Various embodiments of the different aspects of the invention as discussed above are also set out below in the detailed description of the invention, but the invention is not limited thereto. Other aspects of the invention may become apparent from the following description that is given by way of example only and with reference to the accompanying drawings.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

The invention will now be described with reference to the figures in the accompanying drawings.

FIG. 1. Simplified map of the MTB H37Rv reference genome with thirteen gene loci commonly associated with antibiotic resistance labelled. Binding sites for each of the 15 MDA primers are indicated below the genome map.

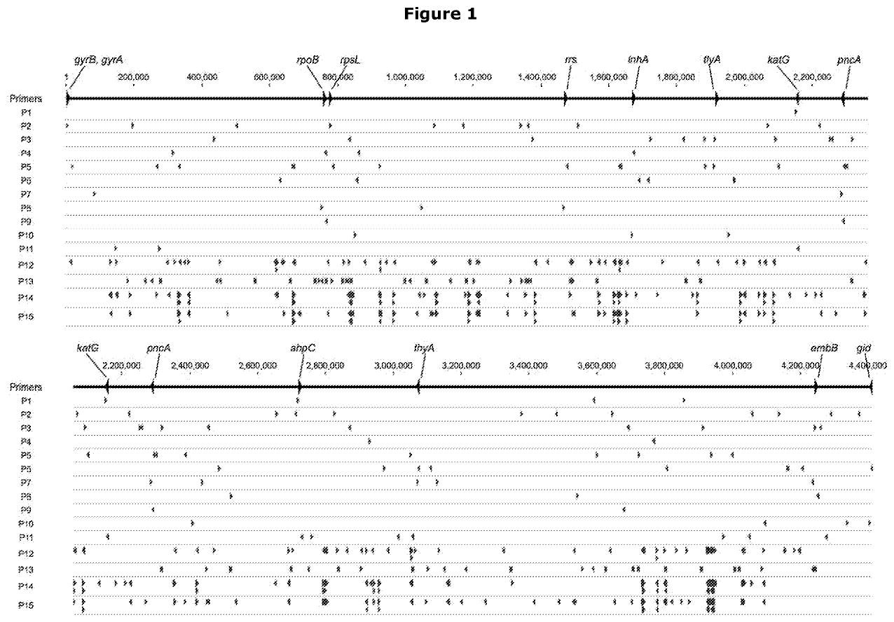

FIG. 2. PerkinElmer LabChip® GX Touch HT analysis of the three targeted MDA reactions using 10-20 ng DNA from sample 5734 as starting template and the non-amplified sample. Left to right, a 40 kb ladder, 16 h MDA for analysis on the Illumina MiSeq, 6 h MDA for analysis on the Illumina MiSeq, 16 h MDA for analysis with the Oxford Nanopore MinION, and non-amplified DNA from the original sample. There are clear differences in the size and quantity of the most common DNA fragments in each sample. These results suggest amplification and concatenation of DNA during the MDA reaction. The greatest difference appears to be between the unamplified and 16 hr MDA; the intensity of the 6 h MDA band is similar to that of the 16 hr samples suggesting strong amplification, however its smaller size suggests concatenation has been more limited.

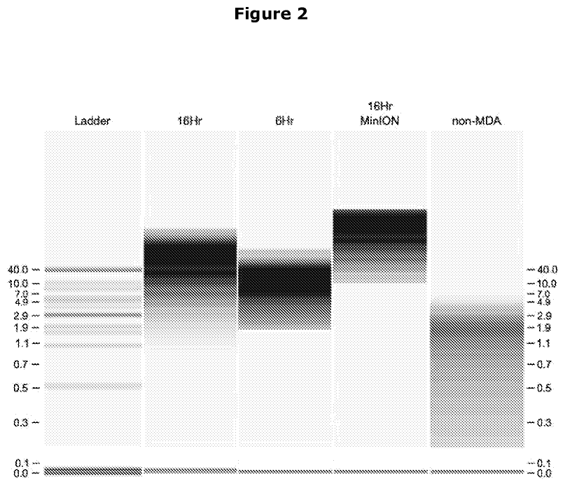

FIG. 3. Quality scores from Illumina MiSeq sequencing of the original non-amplified sample 5734 (A, B are read 1 and read 2, respectively) and targeted MDA reactions of 6 hr (C, D are read 1 and read 2, respectively) and 16 hr (E, F are read 1 and read 2, respectively) using 10-20 ng DNA as starting template. Graphs are from fastQC and show Illumina quality scores for bases 1-151 of sequence reads. In all cases sequencing is of high quality; the vast majority of reads have quality scores of Q30 and above. However, quality scores are higher for read 2 when sequencing MDA templates.

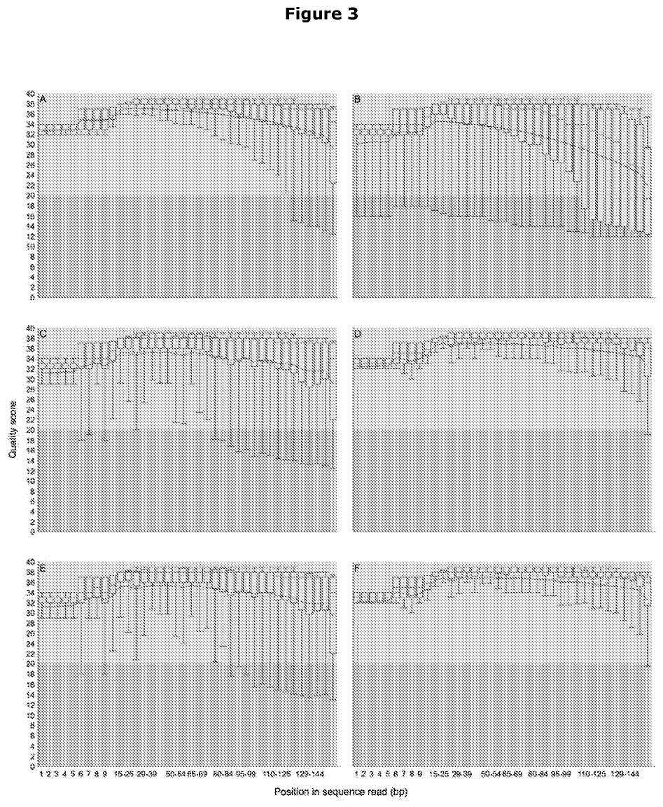

### DETAILED DESCRIPTION OF THE INVENTION

### Definitions

The following definitions are presented to better define the present invention and as a guide for those of ordinary skill in the art in the practice of the present invention.

Unless otherwise specified, all technical and scientific terms used herein are to be understood as having the same meanings as is understood by one of ordinary skill in the relevant art to which this disclosure pertains. Examples of definitions of common terms in microbiology, molecular biology and biochemistry can be found in Methods for General and Molecular Microbiology, 3rd Edition, C. A. Reddy, et al. (eds.), ASM Press, (2008); Encyclopedia of Microbiology, 2nd ed., Joshua Lederburg, (ed.), Academic Press, (2000); Microbiology By Cliffs Notes, I. Edward Alcamo, Wiley, (1996); Dictionary of Microbiology and Molecular Biology, Singleton et al. (2d ed.) (1994); Biology of Microorganisms 11th ed., Brock et al., Pearson Prentice Hall, (2006); Genes IX, Benjamin Lewin, Jones & Bartlett Publishing, (2007); The Encyclopedia of Molecular Biology, Kendrew et al. (eds.), Blackwell Science Ltd., (1994) and Molecular Biology and Biotechnology: a Comprehensive Desk Reference, Robert A. Meyers (ed.), VCH Publishers, Inc., (1995).

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in a similar manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

The term “specifically binds” as used herein with reference to an oligonucleotide primer binding a nucleic acid, particularly DNA, means annealing of the specified primer to portions of the nucleic acid containing a nucleotide sequence complementary to that of the primer. The degree of complementarity between the nucleic acid and the oligonucleotide primer will normally be determined by the conditions under which they come into contact; that is to say the oligonucleotide primer may bind, and thereby allow the initiation of amplification, to portions of the nucleic acid that are at least partially complementary, preferably fully complementary, across the entire length of the primer, or part thereof.

The skilled person in the art will appreciate that in the context of the present invention, a partially complementary oligonucleotide primer can specifically bind to a target nucleic acid to initiate replication under suitable conditions. Accordingly, in some embodiments, an oligonucleotide primer of the invention is partially complementary to the target nucleic acid because it comprises 1, 2 or 3 mismatches, but still specifically binds to a nucleic acid sequence in the M. tuberculosis genome. The phrase, “selectively amplifying” as used herein with reference to a nucleic acid, particularly a DNA, means to preferentially replicate, using a DNA polymerase, the nucleic acids of one of the bacterial species or strains from a sample containing nucleic acids from two or more bacterial species or strains. Related terms such as “selective amplification” and “selectively amplified” are to be interpreted in a similar manner.

The term, “identifying from among the amplified DNA” as used herein refers to bioinformatics analyses of DNA sequences from the amplified DNA that allow the skilled person to determine, given an appropriate set of reference sequences, the likely source of any given DNA sequence from among the amplified DNA.

The term “assign the amplified DNA with high confidence to a particular bacterial species or strain” as used herein means that given the criteria employed by the bioinformatics analyses being used to identify DNA sequences from the amplified DNA, it is much more likely that the given DNA sequence is representative of the indicated genome than any other in the reference set.

The phrases, “linked to, or are directly involved in antibiotic resistance in the species or strain” and “linked to, or are directly involved in antibiotic resistance in M. tuberculosis” and grammatical variations thereof as used herein refer to genes for which mutations have been reported that are assumed, or have been shown, to be responsible for the ability of the bacterial species or strain to survive the application of said antibiotic.

The term “antibiotic resistance profile” as used herein refers to a descriptive listing of the antibiotics that a bacterial species or strain has acquired resistance to.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION

The present invention relates generally to a set of unique oligonucleotide primers that specifically bind the genomic DNA of certain Mycobacterium spp. and allow the selective amplification of Mycobacterium spp. DNA from mixed samples containing DNA from multiple microbial species. The present invention also generally relates to a method of selectively amplifying the DNA of at least one strain of Mycobacterium tuberculosis or M. bovis from a sample using a set of 7 to 12 unique oligonucleotide primers.

Multiple displacement amplification (MDA) is an isothermal approach to enzymatic DNA amplification. Rather than high temperature being used to make the nucleic acid single stranded prior to copying, the enzymes used in MDA reactions have innate strand displacement activity. These enzymes include the bacteriophage 029 DNA polymerase (Pan et al. 2008) and the Bst DNA polymerase (Gadkar et al. 2014). In MDA reactions oligonucleotide primers that bind to multiple sites on the nucleic acid template are used to initiate amplification. As strand synthesis proceeds the polymerase displaces this newly produced DNA strand, which itself becomes a template for further amplification. The process continues and produces a hyper-branched network of double stranded DNA fragments.

The inventors have designed 15 M. tuberculosis-specific oligonucleotide primers as described herein. Of these oligonucleotide primers 11 were selected, in part, because for each the set of possible binding positions on the M. tuberculosis genome included at least one that was within 5 kb, in either the upstream or downstream directions, of one or more of 13 genes linked to or directly involved in antibiotic resistance in M. tuberculosis. More than 30 gene loci have been associated with antibiotic resistance in M. tuberculosis (Dookie et al. 2018), however in more than 95% of clinical cases resistance is associated with one of the 13 targeted genes (Feuerriegel et al. 2015). Use of these oligonucleotide primers—or potentially a subset of 6 thereof—ensures that in subsequent WTS the amplification products of a MDA reaction provide coverage of the 13 genes linked to or directly involved in antibiotic resistance in M. tuberculosis. The remaining four primers were selected primarily because they bind more frequently to the M. tuberculosis genome than to other examined genomes. These latter primers have markedly higher numbers of binding sites on the M. tuberculosis and when used singly, or in combination, thereby increase the overall efficiency of the MDA reaction. The inventors have surprisingly found that the amplification product generated using their inventive primers provide sufficient DNA for robust WGS of M. tuberculosis. The resulting nucleic acids can be analysed to evaluate the DNA sequence of the genome as a whole or of gene loci associated with antibiotic resistance in M. tuberculosis. These data can then be used to provide an antibiotic resistance profile for the sequenced strain.

As typically employed in the art, the goal of MDA is to increase the overall concentration of DNA in a sample. In such cases the oligonucleotide primers often consist of six random nucleotides (so-called “random hexamers”). The expectation is that these hexamers will bind all the nucleic acids in the sample to approximately the same extent and therefore enrichment of DNA in the sample will be unbiased. More recently researchers have begun to use longer oligonucleotide primers that have greater specificity to the genomes of interest (Leichty & Brisson 2014; Clarke et al. 2017). In the context of the present invention, this second approach is used to selectively amplify M. tuberculosis genomic DNA in either the presence (e.g., a sputum sample) or absence (e.g., a young culture) of DNA from other organisms. The selective amplification of M. tuberculosis genomic DNA from a sample containing multiple genomes using MDA requires that the binding sites for the oligonucleotide primers used in the reaction occur more frequently in the genomic sequence of the target organism than in the genomic sequence of any other organism(s) that may be present in the sample (Leichty & Brisson 2014).

To identify suitable oligonucleotide primers the inventors conducted genome-wide “k-mer” (i.e., a nucleotide string of length “k”) searches of M. tuberculosis, human and 15 other organisms commonly found in the human respiratory tract (Table 2). For these analyses, genomes were compared using all k-mers of 6 to 15 nucleotides in length. From these analyses the inventors determined that some oligonucleotide primers 11 and 12 nucleotides in length contain sufficient complexity to selectively amplify M. tuberculosis DNA. In this context both oligonucleotide primer length and base composition are important considerations; other workers have recently developed MDA primers that allow whole genome amplification of M. tuberculosis DNA (Clarke et al. 2017), however these primers are unsuitable for enriching M. tuberculosis from sputum samples because they are shorter and have lower melting temperatures.

The frequency and distribution of specific oligonucleotide primers was then evaluated using the M. tuberculosis H37Rv reference genome. Eleven 12mers were selected, in part, on the basis that for each of these primers the possible binding positions on the M. tuberculosis genome included at least one that was within 5 kb, in either the upstream or downstream directions, of one or more of 13 genes commonly linked to or directly involved in antibiotic resistance in M. tuberculosis. The remaining four primers were selected primarily because they bind more frequently to the M. tuberculosis genome than to other examined genomes. In some embodiments, primer sets including fewer than 15 oligonucleotide primers may be employed.

A skilled worker using the primers and methods described herein is provided with a number of unexpected advantages over other cell-free methods of detecting M. tuberculosis and of profiling the antibiotic resistance capabilities of the detected bacteria currently known in the art. For example, quite importantly and quite unexpectedly, the inventors have found that conducting MDA at higher temperatures than typically used, increases the specificity for selectively amplifying Mycobacterium tuberculosis when present in complex mixtures of micro-organisms such as occurs with sputum.

Accordingly, in one aspect the invention relates to a composition comprising 7 to 12 unique oligonucleotide primers, each primer consisting of 11 or 12 nucleotides, wherein each of these oligonucleotide primers specifically binds to a nucleic acid sequence in the M. tuberculosis genome.

In one embodiment the composition comprises 7 to 15 unique oligonucleotide primers.

In one embodiment the composition consists essentially of the unique oligonucleotide primers.

In one embodiment each of the oligonucleotide primers selectively binds no more than 12 kb away, preferably no more than 10 kb away, preferably no more than 8 kb, preferably no more than 5 kb away, preferably no more than 1 kb away from a gene locus in the M. tuberculosis genome that is linked to, or that is directly involved with, antibiotic resistance.

In one embodiment each of the oligonucleotide primers selectively binds about 12 kb or less, preferably about 10 kb or less, preferably about 8 kb or less, preferably about 5 kb or less, preferably about 1 kb or less from a gene locus in the M. tuberculosis genome that is linked to, or that is directly involved with, antibiotic resistance.

In one embodiment the gene locus is selected from the group consisting of alkyl hydroperoxidase reductase subunit C (ahpC), arabinosyl transferase B (embB), 7-methylguanosine methyltransferase (gidB), DNA gyrase (gyrA), DNA gyrase (gyrB), NADH-dependent enoyl-acyl carrier protein reductase (inhA), catalase/peroxidase (katG), pyrazinamidase/nicotinamidase (pncA), RNA polymerase β subunit (rpoB), ribosomal protein S12 (rpsL), 16S rRNA (rrs), Thymidylate synthase (thyA) and rRNA methyltransferase (tlyA).

In one embodiment, each of the oligonucleotide primers selectively binds within 12 kb, preferably within 10 kb, preferably within 8 kb, preferably within 5 kb, of the each of alkyl hydroperoxidase reductase subunit C (ahpC), arabinosyl transferase B (embB), 7-methylguanosine methyltransferase (gidB), DNA gyrase (gyrA), DNA gyrase (gyrB), NADH-dependent enoyl-acyl carrier protein reductase (inhA), catalase/peroxidase (katG), pyrazinamidase/nicotinamidase (pncA), RNA polymerase β subunit (rpoB), ribosomal protein S12 (rpsL), 16S rRNA (rrs), thymidylate synthase (thyA) and rRNA methyltransferase (tlyA).

In one embodiment the oligonucleotide primers are selected from the group consisting of P1 (SEQ ID NO: 1), P2 (SEQ ID NO: 2), P3 (SEQ ID NO: 3), P4 (SEQ ID NO: 4), P5 (SEQ ID NO: 5), P6 (SEQ ID NO: 6), P7 (SEQ ID NO: 7), P8 (SEQ ID NO: 8), P9 (SEQ ID NO: 9), P10 (SEQ ID NO: 10), P11 (SEQ ID NO: 11), P12 (SEQ ID NO: 12), P13 (SEQ ID NO: 13), P14 (SEQ ID NO: 14) and P15 (SEQ ID NO: 15).

In one embodiment the composition comprises P1-P6 and at least one of, preferably at least two of, preferably at least three of, preferably all four of P12, P13, P14 or P15.

In one embodiment the composition consists essentially of P1-P6 and at least one of, preferably at least two of, preferably at least three of, preferably all four of P12, P13, P14 or P15.

In one embodiment the composition comprises P1-P9 and at least one of, preferably at least two of, preferably at least three of, preferably all four of P12, P13, P14 or P15.

In one embodiment the composition consists essentially of P1-P9 and at least one of, preferably at least two of, preferably at least three of, preferably all four of P12, P13, P14 or P15.

In one embodiment the composition comprises P1-P11 and at least one of, preferably at least two of, preferably at least three of, preferably all four of P12, P13, P14 or P15.

In one embodiment the composition consists essentially of P1-P11 and at least one of, preferably at least two of, preferably at least three of, preferably all four of P12, P13, P14 or P15.

In one embodiment the composition comprises P1-P6 and P12. In one embodiment the composition comprises P1-P6 and P13. In one embodiment the composition comprises P1-P6 and P14. In one embodiment the composition comprises P1-P6 and P15. In one embodiment the composition comprises P1-P6, P12 and P13. In one embodiment the composition comprises P1-P6, P12 and P14. In one embodiment the composition comprises P1-P6, P12 and P15. In one embodiment the composition comprises P1-P6, P13 and P14. In one embodiment the composition comprises P1-P6, P13 and P15. In one embodiment the composition comprises P1-P6, P14 and P15. In one embodiment the composition comprises P1-P6, P12, P13 and P14. In one embodiment the composition comprises P1-P6, P12, P13 and P15. In one embodiment the composition comprises P1-P6, P12, P14 and P15. In one embodiment the composition comprises P1-P6, P13, P14 and P15. In one embodiment the composition comprises P1-P6, P12, P13, P14 and P15. In one embodiment the composition comprises P1-P9 and P12. In one embodiment the composition comprises P1-P9 and P13. In one embodiment the composition comprises P1-P9 and P14. In one embodiment the composition comprises P1-P9 and P15. In one embodiment the composition comprises P1-P9, P12 and P13. In one embodiment the composition comprises P1-P9, P12 and P14. In one embodiment the composition comprises P1-P9, P12 and P15. In one embodiment the composition comprises P1-P9, P13 and P14. In one embodiment the composition comprises P1-P9, P13 and P15. In one embodiment the composition comprises P1-P9, P14 and P15. In one embodiment the composition comprises P1-P9, P12, P13 and P14. In one embodiment the composition comprises P1-P9, P12, P13 and P15. In one embodiment the composition comprises P1-P9, P12, P14 and P15. In one embodiment the composition comprises P1-P9, P13, P14 and P15. In one embodiment the composition comprises P1-P9, P12, P13, P14 and P15. In one embodiment the composition comprises P1-P12. In one embodiment the composition comprises P1-P11 and P13. In one embodiment the composition comprises P1-P11 and P14. In one embodiment the composition comprises P1-P11 and P15. In one embodiment the composition comprises P1-P12 and P13. In one embodiment the composition comprises P1-P12 and P14. In one embodiment the composition comprises P1-P12 and P15. In one embodiment the composition comprises P1-P11, P13 and P14. In one embodiment the composition comprises P1-P11, P13 and P15. In one embodiment the composition comprises P1-P11, P14 and P15. In one embodiment the composition comprises P1-P12, P13 and P14. In one embodiment the composition comprises P1-P12, P13 and P15. In one embodiment the composition comprises P1-P12, P14 and P15. In one embodiment the composition comprises P1-P11, P13, P14 and P15. In one embodiment the composition comprises P1-P15.

In one embodiment the composition consists essentially of P1-P6 and P12. In one embodiment the composition consists essentially of P1-P6 and P13. In one embodiment the composition consists essentially of P1-P6 and P14. In one embodiment the composition consists essentially of P1-P6 and P15. In one embodiment the composition consists essentially of P1-P6, P12 and P13. In one embodiment the composition consists essentially of P1-P6, P12 and P14. In one embodiment the composition consists essentially of P1-P6, P12 and P15. In one embodiment the composition consists essentially of P1-P6, P13 and P14. In one embodiment the composition consists essentially of P1-P6, P13 and P15. In one embodiment the composition consists essentially of P1-P6, P14 and P15. In one embodiment the composition consists essentially of P1-P6, P12, P13 and P14. In one embodiment the composition consists essentially of P1-P6, P12, P13 and P15. In one embodiment the composition consists essentially of P1-P6, P12, P14 and P15. In one embodiment the composition consists essentially of P1-P6, P13, P14 and P15. In one embodiment the composition consists essentially of P1-P6, P12, P13, P14 and P15. In one embodiment the composition consists essentially of P1-P9 and P12. In one embodiment the composition consists essentially of P1-P9 and P13. In one embodiment the composition consists essentially of P1-P9 and P14. In one embodiment the composition consists essentially of P1-P9 and P15. In one embodiment the composition consists essentially of P1-P9, P12 and P13. In one embodiment the composition consists essentially of P1-P9, P12 and P14. In one embodiment the composition consists essentially of P1-P9, P12 and P15. In one embodiment the composition consists essentially of P1-P9, P13 and P14. In one embodiment the composition consists essentially of P1-P9, P13 and P15. In one embodiment the composition consists essentially of P1-P9, P14 and P15. In one embodiment the composition consists essentially of P1-P9, P12, P13 and P14. In one embodiment the composition consists essentially of P1-P9, P12, P13 and P15. In one embodiment the composition consists essentially of P1-P9, P12, P14 and P15. In one embodiment the composition consists essentially of P1-P9, P13, P14 and P15. In one embodiment the composition consists essentially of P1-P9, P12, P13, P14 and P15. In one embodiment the composition consists essentially of P1-P12. In one embodiment the composition consists essentially of P1-P11 and P13. In one embodiment the composition consists essentially of P1-P11 and P14. In one embodiment the composition consists essentially of P1-P11 and P15. In one embodiment the composition consists essentially of P1-P12 and P13. In one embodiment the composition consists essentially of P1-P12 and P14. In one embodiment the composition consists essentially of P1-P12 and P15. In one embodiment the composition consists essentially of P1-P11, P13 and P14. In one embodiment the composition consists essentially of P1-P11, P13 and P15. In one embodiment the composition consists essentially of P1-P11, P14 and P15. In one embodiment the composition consists essentially of P1-P12, P13 and P14. In one embodiment the composition consists essentially of P1-P12, P13 and P15. In one embodiment the composition consists essentially of P1-P12, P14 and P15. In one embodiment the composition consists essentially of P1-P11, P13, P14 and P15. In one embodiment the composition consists essentially of P1-P15.

The nucleotide sequences of P1-P15 are shown in Table 1.

TABLE 1
M. tuberculosis oligonucleotide primers

| | Primer | Nucleic acid |
|---|---|---|
| SEQ ID NO | Name | sequence (5′-3′) |
| SEQ ID NO: 1 | P1 | AATGGCCGTCGC |
| SEQ ID NO: 2 | P2 | GGTCGGTGCGGG |
| SEQ ID NO: 3 | P3 | TGGCCGGGGTGT |
| SEQ ID NO: 4 | P4 | GCAACACCGGGT |
| SEQ ID NO: 5 | P5 | GCGGGCACGGTG |
| SEQ ID NO: 6 | P6 | CGTCGGCTGCGG |
| SEQ ID NO: 7 | P7 | CCACCCGCGCAA |
| SEQ ID NO: 8 | P8 | GACGCGCCCACG |
| SEQ ID NO: 9 | P9 | TCGCTACCCACG |
| SEQ ID NO: 10 | P10 | ATGTTGGTGATC |
| SEQ ID NO: 11 | P11 | GGTGTCGACGAG |
| SEQ ID NO: 12 | P12 | CGGCGACGGCGG |
| SEQ ID NO: 13 | P13 | TGCGTCTGCTCG |
| SEQ ID NO: 14 | P14 | CCGCCGTTGCC |
| SEQ ID NO: 15 | P15 | CCGTTGCCGCC |

In another aspect the present invention relates to a composition comprising at least 7 unique oligonucleotide primers selected from the group consisting of P1 (SEQ ID NO: 1), P2 (SEQ ID NO: 2), P3 (SEQ ID NO: 3), P4 (SEQ ID NO: 4), P5 (SEQ ID NO: 5), P6 (SEQ ID NO: 6), P7 (SEQ ID NO: 7), P8 (SEQ ID NO: 8), P9 (SEQ ID NO: 9), P10 (SEQ ID NO: 10), P11 (SEQ ID NO: 11), P12 (SEQ ID NO: 12), P13 (SEQ ID NO: 13), P14 (SEQ ID NO: 14) and P15 (SEQ ID NO: 15).

In one embodiment the composition comprises P1-P6 and at least one of, preferably at least two of, preferably at least three of, preferably all four of P12, P13, P14 or P15.

In one embodiment the composition consists essentially of P1-P6 and at least one of, preferably at least two of, preferably at least three of, preferably all four of P12, P13, P14 or P15.

In one embodiment the composition comprises P1-P9 and at least one of, preferably at least two of, preferably at least three of, preferably all four of P12, P13, P14 or P15.

In one embodiment the composition consists essentially of P1-P9 and at least one of, preferably at least two of, preferably at least three of, preferably all four of P12, P13, P14 or P15.

In one embodiment the composition comprises P1-P11 and at least one of, preferably at least two of, preferably at least three of, preferably all four of P12, P13, P14 or P15.

In one embodiment the composition consists essentially of P1-P11 and at least one of, preferably at least two of, preferably at least three of, preferably all four of P12, P13, P14 or P15.

In one embodiment the composition comprises P1-P6 and P12. In one embodiment the composition comprises P1-P6 and P13. In one embodiment the composition comprises P1-P6 and P14. In one embodiment the composition comprises P1-P6 and P15. In one embodiment the composition comprises P1-P6, P12 and P13. In one embodiment the composition comprises P1-P6, P12 and P14. In one embodiment the composition comprises P1-P6, P12 and P15. In one embodiment the composition comprises P1-P6, P13 and P14. In one embodiment the composition comprises P1-P6, P13 and P15. In one embodiment the composition comprises P1-P6, P14 and P15. In one embodiment the composition comprises P1-P6, P12, P13 and P14. In one embodiment the composition comprises P1-P6, P12, P13 and P15. In one embodiment the composition comprises P1-P6, P12, P14 and P15. In one embodiment the composition comprises P1-P6, P13, P14 and P15. In one embodiment the composition comprises P1-P6, P12, P13, P14 and P15. In one embodiment the composition comprises P1-P9 and P12. In one embodiment the composition comprises P1-P9 and P13. In one embodiment the composition comprises P1-P9 and P14. In one embodiment the composition comprises P1-P9 and P15. In one embodiment the composition comprises P1-P9, P12 and P13. In one embodiment the composition comprises P1-P9, P12 and P14. In one embodiment the composition comprises P1-P9, P12 and P15. In one embodiment the composition comprises P1-P9, P13 and P14. In one embodiment the composition comprises P1-P9, P13 and P15. In one embodiment the composition comprises P1-P9, P14 and P15. In one embodiment the composition comprises P1-P9, P12, P13 and P14. In one embodiment the composition comprises P1-P9, P12, P13 and P15. In one embodiment the composition comprises P1-P9, P12, P14 and P15. In one embodiment the composition comprises P1-P9, P13, P14 and P15. In one embodiment the composition comprises P1-P9, P12, P13, P14 and P15. In one embodiment the composition comprises P1-P12. In one embodiment the composition comprises P1-P11 and P13. In one embodiment the composition comprises P1-P11 and P14. In one embodiment the composition comprises P1-P11 and P15. In one embodiment the composition comprises P1-P12 and P13. In one embodiment the composition comprises P1-P12 and P14. In one embodiment the composition comprises P1-P12 and P15. In one embodiment the composition comprises P1-P11, P13 and P14. In one embodiment the composition comprises P1-P11, P13 and P15. In one embodiment the composition comprises P1-P11, P14 and P15. In one embodiment the composition comprises P1-P12, P13 and P14. In one embodiment the composition comprises P1-P12, P13 and P15. In one embodiment the composition comprises P1-P12, P14 and P15. In one embodiment the composition comprises P1-P11, P13, P14 and P15. In one embodiment the composition comprises P1-P15.

In one embodiment the composition consists essentially of P1-P6 and P12. In one embodiment the composition consists essentially of P1-P6 and P13. In one embodiment the composition consists essentially of P1-P6 and P14. In one embodiment the composition consists essentially of P1-P6 and P15. In one embodiment the composition consists essentially of P1-P6, P12 and P13. In one embodiment the composition consists essentially of P1-P6, P12 and P14. In one embodiment the composition consists essentially of P1-P6, P12 and P15. In one embodiment the composition consists essentially of P1-P6, P13 and P14. In one embodiment the composition consists essentially of P1-P6, P13 and P15. In one embodiment the composition consists essentially of P1-P6, P14 and P15. In one embodiment the composition consists essentially of P1-P6, P12, P13 and P14. In one embodiment the composition consists essentially of P1-P6, P12, P13 and P15. In one embodiment the composition consists essentially of P1-P6, P12, P14 and P15. In one embodiment the composition consists essentially of P1-P6, P13, P14 and P15. In one embodiment the composition consists essentially of P1-P6, P12, P13, P14 and P15. In one embodiment the composition consists essentially of P1-P9 and P12. In one embodiment the composition consists essentially of P1-P9 and P13. In one embodiment the composition consists essentially of P1-P9 and P14. In one embodiment the composition consists essentially of P1-P9 and P15. In one embodiment the composition consists essentially of P1-P9, P12 and P13. In one embodiment the composition consists essentially of P1-P9, P12 and P14. In one embodiment the composition consists essentially of P1-P9, P12 and P15. In one embodiment the composition consists essentially of P1-P9, P13 and P14. In one embodiment the composition consists essentially of P1-P9, P13 and P15. In one embodiment the composition consists essentially of P1-P9, P14 and P15. In one embodiment the composition consists essentially of P1-P9, P12, P13 and P14. In one embodiment the composition consists essentially of P1-P9, P12, P13 and P15. In one embodiment the composition consists essentially of P1-P9, P12, P14 and P15. In one embodiment the composition consists essentially of P1-P9, P13, P14 and P15. In one embodiment the composition consists essentially of P1-P9, P12, P13, P14 and P15. In one embodiment the composition consists essentially of P1-P12. In one embodiment the composition consists essentially of P1-P11 and P13. In one embodiment the composition consists essentially of P1-P11 and P14. In one embodiment the composition consists essentially of P1-P11 and P15. In one embodiment the composition consists essentially of P1-P12 and P13. In one embodiment the composition consists essentially of P1-P12 and P14. In one embodiment the composition consists essentially of P1-P12 and P15. In one embodiment the composition consists essentially of P1-P11, P13 and P14. In one embodiment the composition consists essentially of P1-P11, P13 and P15. In one embodiment the composition consists essentially of P1-P11, P14 and P15. In one embodiment the composition consists essentially of P1-P12, P13 and P14. In one embodiment the composition consists essentially of P1-P12, P13 and P15. In one embodiment the composition consists essentially of P1-P12, P14 and P15. In one embodiment the composition consists essentially of P1-P11, P13, P14 and P15. In one embodiment the composition consists essentially of P1-P15.

In one embodiment the composition further comprises at least one enzyme that catalyzes nucleic acid replication. In one embodiment the enzyme is a 029 polymerase. In one embodiment the enzyme is a Bst polymersase.

In another aspect the invention relates to a kit comprising at least 7 unique oligonucleotide primers selected from the group consisting of P1-P14 and P15, and at least one enzyme that catalyzes nucleic acid replication.

In one embodiment the enzyme is a 029 polymerase.

In one embodiment the enzyme is a Bst polymersase.

In one embodiment the kit comprises P1-P6 and P12. In one embodiment the kit comprises P1-P6 and P13. In one embodiment the kit comprises P1-P6 and P14. In one embodiment the kit comprises P1-P6 and P15. In one embodiment the kit comprises P1-P6, P12 and P13. In one embodiment the kit comprises P1-P6, P12 and P14. In one embodiment the kit comprises P1-P6, P12 and P15. In one embodiment the kit comprises P1-P6, P13 and P14. In one embodiment the kit comprises P1-P6, P13 and P15. In one embodiment the kit comprises P1-P6, P14 and P15. In one embodiment the kit comprises P1-P6, P12, P13 and P14. In one embodiment the kit comprises P1-P6, P12, P13 and P15. In one embodiment the kit comprises P1-P6, P12, P14 and P15. In one embodiment the kit comprises P1-P6, P13, P14 and P15. In one embodiment the kit comprises P1-P6, P12, P13, P14 and P15. In one embodiment the kit comprises P1-P9 and P12. In one embodiment the kit comprises P1-P9 and P13. In one embodiment the kit comprises P1-P9 and P14. In one embodiment the kit comprises P1-P9 and P15. In one embodiment the kit comprises P1-P9, P12 and P13. In one embodiment the kit comprises P1-P9, P12 and P14. In one embodiment the kit comprises P1-P9, P12 and P15. In one embodiment the kit comprises P1-P9, P13 and P14. In one embodiment the kit comprises P1-P9, P13 and P15. In one embodiment the kit comprises P1-P9, P14 and P15. In one embodiment the kit comprises P1-P9, P12, P13 and P14. In one embodiment the kit comprises P1-P9, P12, P13 and P15. In one embodiment the kit comprises P1-P9, P12, P14 and P15. In one embodiment the kit comprises P1-P9, P13, P14 and P15. In one embodiment the kit comprises P1-P9, P12, P13, P14 and P15. In one embodiment the kit comprises P1-P12. In one embodiment the kit comprises P1-P11 and P13. In one embodiment the kit comprises P1-P11 and P14. In one embodiment the kit comprises P1-P11 and P15. In one embodiment the kit comprises P1-P12 and P13. In one embodiment the kit comprises P1-P12 and P14. In one embodiment the kit comprises P1-P12 and P15. In one embodiment the kit comprises P1-P11, P13 and P14. In one embodiment the kit comprises P1-P11, P13 and P15. In one embodiment the kit comprises P1-P11, P14 and P15. In one embodiment the kit comprises P1-P12, P13 and P14. In one embodiment the kit comprises P1-P12, P13 and P15. In one embodiment the kit comprises P1-P12, P14 and P15. In one embodiment the kit comprises P1-P11, P13, P14 and P15. In one embodiment the kit comprises P1-P15.

In one embodiment the kit consists essentially of P1-P6 and P12. In one embodiment the kit consists essentially of P1-P6 and P13. In one embodiment the kit consists essentially of P1-P6 and P14. In one embodiment the kit consists essentially of P1-P6 and P15. In one embodiment the kit consists essentially of P1-P6, P12 and P13. In one embodiment the kit consists essentially of P1-P6, P12 and P14. In one embodiment the kit consists essentially of P1-P6, P12 and P15. In one embodiment the kit consists essentially of P1-P6, P13 and P14. In one embodiment the kit consists essentially of P1-P6, P13 and P15. In one embodiment the kit consists essentially of P1-P6, P14 and P15. In one embodiment the kit consists essentially of P1-P6, P12, P13 and P14. In one embodiment the kit consists essentially of P1-P6, P12, P13 and P15. In one embodiment the kit consists essentially of P1-P6, P12, P14 and P15. In one embodiment the kit consists essentially of P1-P6, P13, P14 and P15. In one embodiment the kit consists essentially of P1-P6, P12, P13, P14 and P15. In one embodiment the kit consists essentially of P1-P9 and P12. In one embodiment the kit consists essentially of P1-P9 and P13. In one embodiment the kit consists essentially of P1-P9 and P14. In one embodiment the kit consists essentially of P1-P9 and P15. In one embodiment the kit consists essentially of P1-P9, P12 and P13. In one embodiment the kit consists essentially of P1-P9, P12 and P14. In one embodiment the kit consists essentially of P1-P9, P12 and P15. In one embodiment the kit consists essentially of P1-P9, P13 and P14. In one embodiment the kit consists essentially of P1-P9, P13 and P15. In one embodiment the kit consists essentially of P1-P9, P14 and P15. In one embodiment the kit consists essentially of P1-P9, P12, P13 and P14. In one embodiment the kit consists essentially of P1-P9, P12, P13 and P15. In one embodiment the kit consists essentially of P1-P9, P12, P14 and P15. In one embodiment the kit consists essentially of P1-P9, P13, P14 and P15. In one embodiment the kit consists essentially of P1-P9, P12, P13, P14 and P15. In one embodiment the kit consists essentially of P1-P12. In one embodiment the kit consists essentially of P1-P11 and P13. In one embodiment the kit consists essentially of P1-P11 and P14. In one embodiment the kit consists essentially of P1-P11 and P15. In one embodiment the kit consists essentially of P1-P12 and P13. In one embodiment the kit consists essentially of P1-P12 and P14. In one embodiment the kit consists essentially of P1-P12 and P15. In one embodiment the kit consists essentially of P1-P11, P13 and P14. In one embodiment the kit consists essentially of P1-P11, P13 and P15. In one embodiment the kit consists essentially of P1-P11, P14 and P15. In one embodiment the kit consists essentially of P1-P12, P13 and P14. In one embodiment the kit consists essentially of P1-P12, P13 and P15. In one embodiment the kit consists essentially of P1-P12, P14 and P15. In one embodiment the kit consists essentially of P1-P11, P13, P14 and P15. In one embodiment the kit consists essentially of P1-P15.

In another aspect the invention relates to a method of selectively amplifying the genomic DNA of at least one bacterial species or strain from a sample, the method comprising:
contacting the sample with a composition comprising 7-12 unique oligonucleotide primers, each primer consisting of 11 or 12 nucleotides, wherein each of these oligonucleotide primers specifically binds to a nucleic acid sequence in the genome of the bacterial species or strain,
selectively amplifying DNA from the bacterial species or strain of interest in a multiple displacement amplification (MDA) reaction,
identifying from among the selectively amplified DNA, DNA sequences that are assigned with high confidence to the genome of the bacterial species or strain of interest.

In one embodiment the composition comprises 7 to 15 unique oligonucleotide primers.

Specifically contemplated as embodiments of this aspect of the invention that is a method of selectively amplifying the genomic DNA of at least one bacterial species or strain are all of the embodiments of the invention set forth in the composition aspects of the invention, including the use of the unique oligonucleotide primers and combinations of unique oligonucleotide primers set forth in the composition aspects and embodiments of the invention.

In one embodiment identifying comprises sequencing and bioinformatics analysis of the amplified DNA products.

In one embodiment the DNA sequences are identified as encoding bacterial gene products that are linked to or directly involved in conferring antibiotic resistance in the at least one bacterial species or strain.

In one embodiment the DNA sequences are identified as encoding proteins or portions thereof or RNAs or portions thereof that are linked to or directly involved in conferring antibiotic resistance in the at least one bacterial species or strain.

In one embodiment, identifying from among the selectively amplified DNA, DNA sequences that are assigned with high confidence to the genome of a bacterial species or strain of interest comprises whole genome sequencing (WGS) of the amplified DNA and bioinformatics analysis of the obtained nucleotide sequences to determine the nucleotide sequence of the at least one bacterial species or strain.

In one embodiment, the method further comprises identifying from among the selectively amplified DNA, DNA sequences encoding bacterial gene products that are linked to or directly involved in conferring antibiotic resistance in the bacterial species or strain comprising generating an antibiotic resistance profile by whole genome sequencing (WGS) and bioinformatics analysis of the amplified DNA to determine the nucleotide sequence of at least one gene locus that is linked to or that is directly involved in antibiotic resistance in the bacterial species or strain.

In one embodiment, generating the antibiotic resistance profile comprises WGS and bioinformatics analysis of the amplified DNA to determine the nucleotide sequence of at least one, preferably at least two, preferably at least 3, preferably at least 4, preferably at least 5, preferably at least 6, preferably at least 7, preferably at least 8, preferably at least 9, preferably at least 10, preferably at least 11, preferably at least 12, preferably 13 gene loci that are linked to or that are directly involved in antibiotic resistance in the at least one bacterial species or strain.

In one embodiment the gene loci are selected from the group consisting of alkyl hydroperoxidase reductase subunit C (ahpC), arabinosyl transferase B (embB), 7-methylguanosine methyltransferase (gidB), DNA gyrase (gyrA), DNA gyrase (gyrB), NADH-dependent enoyl-acyl carrier protein reductase (inhA), catalase/peroxidase (katG), pyrazinamidase/nicotinamidase (pncA), RNA polymerase β subunit (rpoB), ribosomal protein S12 (rpsL), 16S rRNA (rrs), thymidylate synthase (thyA) and rRNA methyltransferase (tlyA).

In some embodiments the method comprises identifying an antibiotic that is effective against the at least one bacterial species or strain based on the antibiotic resistance profile.

In one embodiment the at least one bacterial species or strain is Mycobacterium. In one embodiment the Mycobacterium is Mycobacterium tuberculosis or Mycobacterium bovis.

In one embodiment the sample is a culture of M. tuberculosis or M. bovis.

In one embodiment the culture is less than 5 days, preferably less than 4 days, preferably less than 3 days old.

In one embodiment the MDA reaction is carried out using a 029 polymerase.

In one embodiment the MDA reaction is carried out at a first temperature of about 25° C. to about 40° C., preferably about 26° C. to about 38° C., preferably about 27° C. to about 36° C., preferably about 28° C. to about 34° C., preferably about 29° C. to about 32° C., preferably about 30° C.

In one embodiment the MDA reaction is carried out at a first temperature of about 25° C. to about 45° C., preferably about 30° C. to about 45° C., preferably about 30° C. to about 44° C., preferably about 32° C. to about 43° C., preferably about 33° C. to about 42° C., preferably about 34° C. to about 41° C., preferably about 35° C. to about 40° C., preferably about 36° C. to about 39° C., preferably about 36° C. to about 38° C., preferably about 37° C.

In one embodiment the MDA reaction is carried out at a first temperature of 25° C. to 40° C., preferably 26° C. to 38° C., preferably 27° C. to 36° C., preferably 28° C. to 34° C., preferably 29° C. to 32° C., preferably 30° C.

In one embodiment the MDA reaction is carried out at a first temperature of 25° C. to 45° C., preferably 30° C. to 45° C., preferably 30° C. to 44° C., preferably 32° C. to 43° C., preferably 33° C. to 42° C., preferably 34° C. to 41° C., preferably 35° C. to 40° C., preferably 36° C. to 39° C., preferably 36° C. to 38° C., preferably 37° C.

In one embodiment the MDA reaction is incubated at a first temperature for at least 1 hour, preferably for at least 2 h, preferably for at least 3 h, preferably for at least 4 h, preferably for at least 5 h, preferably for at least 6 h, preferably for at least 7 h, preferably for at least 8 h, preferably for at least 9 h, preferably for at least 10 h, preferably for at least 11 h, preferably for at least 12 h, preferably for at least 13 h, preferably for at least 14 h, preferably for at least 15 h, preferably for at least 16 h.

In one embodiment the MDA reaction is carried out at a first temperature for up to 1 hour, preferably for up to 2 h, preferably for up to 3 h, preferably for up to 4 h, preferably for up to 5 h, preferably for up to 6 h, preferably for up to 7 h, preferably for up to 8 h, preferably for up to 9 h, preferably for up to 10 h, preferably for up to 11 h, preferably for up to 12 h, preferably for up to 13 h, preferably for up to 14 h, preferably for up to 15 h, preferably for up to 16 h.

In one embodiment the MDA reaction is carried out at a first temperature for about 1 hour, preferably for about 2 h, preferably for about 3 h, preferably for about 4 h, preferably for about 5 h, preferably for about 6 h, preferably for about 7 h, preferably for about 8 h, preferably for about 9 h, preferably for about 10 h, preferably for about 11 h, preferably for about 12 h, preferably for about 13 h, preferably for about 14 h, preferably for about 15 h, preferably for about 16 h.

In one embodiment the MDA reaction is carried out at a first temperature of about 25° C. to about 40° C., preferably about 26° C. to about 38° C., preferably about 27° C. to about 36° C., preferably about 28° C. to about 34° C., preferably about 29° C. to about 32° C., preferably about 30° C.

In one embodiment the MDA reaction is carried out at a first temperature of about 25° C. to about 45° C., preferably about 30° C. to about 45° C., preferably about 30° C. to about 44° C., preferably about 32° C. to about 43° C., preferably about 33° C. to about 42° C., preferably about 34° C. to about 41° C., preferably about 35° C. to about 40° C., preferably about 36° C. to about 39° C., preferably about 36° C. to about 38° C., preferably about 37° C.

In one embodiment the MDA reaction is carried out at a first temperature of 25° C. to 40° C., preferably 26° C. to 38° C., preferably 27° C. to 36° C., preferably 28° C. to 34° C., preferably 29° C. to 32° C., preferably 30° C.

In one embodiment the MDA reaction is carried out at a first temperature of 25° C. to 45° C., preferably 30° C. to 45° C., preferably 30° C. to 44° C., preferably 32° C. to 43° C., preferably 33° C. to 42° C., preferably 34° C. to 41° C., preferably 35° C. to 40° C., preferably 36° C. to 39° C., preferably 36° C. to 38° C., preferably 37° C.

In one embodiment the MDA reaction is further incubated at a second temperature for about 10 min, preferably 10 min.

In one embodiment the second temperature is at about 65° C., preferably is at 65° C.

In one embodiment the MDA reaction comprises a buffer, deoxyribonucleotide triphosphates, bovine serum albumin and trehalose-dihydrate. In one embodiment the MDA reaction comprises yeast inorganic pyrophosphatase and/or potassium chloride.

In one embodiment the MDA reaction contains less than 30 ng, preferably less than 15 ng, preferably less than 10 ng, preferably less than 5 ng, preferably less than 2 ng, preferably less than 0.2 ng, preferably less than 0.02 ng, preferably less than 0.002 ng of M. tuberculosis or M. bovis DNA.

In one embodiment the sample is a sample containing or suspected of containing DNA from M. tuberculosis or M. bovis, and DNA from at least one other organism, preferably from at least 2, preferably from at least 5, preferably from at least 10, preferably from at least 15 other organisms.

In one embodiment the at least one other organism is selected from the group consisting of prokaryotes and eukaryotes. In one embodiment the prokaryotes are bacteria. In one embodiment the bacterial are Gram-negative or Gram-positive bacteria, or both.

In one embodiment the eukaryotes are protists or animals.

In one embodiment the animals are mammals.

In one embodiment the mammals are selected from the group consisting of humans, bovines, ovines, cervines, canines, felines, porcines, and camelids.

In one embodiment the sample contains or also contains, DNA or RNA from a virus.

In one embodiment the sample is from a human.

In one embodiment the sample is from a cow.

In one embodiment the sample is a sputum sample.

In one embodiment the sample is a saliva sample.

In this embodiment the MDA reaction is carried out using a Bst polymerase.

In one embodiment the MDA reaction is carried out at a first temperature of about 38° C. to about 60° C., preferably about 40° C. to about 56° C., preferably about 42° C. to about 52° C., preferably about 44° C. to about 48° C., preferably about 45° C., preferably about 46° C., preferably about 47° C.

In one embodiment the MDA reaction is carried out at a first temperature of 38° C. to 60° C., preferably 40° C. to 56° C., preferably 42° C. to 52° C., preferably 44° C. to aout 48° C., preferably 45° C., preferably 46° C., preferably 47° C.

In one embodiment the MDA reaction is incubated at a first temperature for at least 1 hour, preferably for at least 2 h, preferably for at least 3 h, preferably for at least 4 h, preferably for at least 5 h, preferably for at least 6 h.

In one embodiment the MDA reaction is carried out at a first temperature for up to 1 hour, preferably for up to 2 h, preferably for up to 3 h, preferably for up to 4 h, preferably for up to 5 h, preferably for up to 6 h.

In one embodiment the MDA reaction is carried out at a first temperature for about 1 hour, preferably for about 2 h, preferably for about 3 h, preferably for about 4 h, preferably for about 5 h, preferably for about 6 h.

In one embodiment the MDA reaction is carried out at a first temperature for 1 hour, preferably for 2 h, preferably for 3 h, preferably for 4 h, preferably for 5 h, preferably for 6 h.

In one embodiment the MDA reaction is carried out at a first temperature of about 38° C. to about 60° C., preferably about 40° C. to about 56° C., preferably about 42° C. to about 52° C., preferably about 44° C. to about 48° C., preferably about 45° C., preferably about 46° C., preferably about 47° C.

In one embodiment the MDA reaction is carried out at a first temperature of 38° C. to 60° C., preferably 40° C. to 56° C., preferably 42° C. to 52° C., preferably 44° C. to about 48° C., preferably 45° C., preferably 46° C., preferably 47° C.

In one embodiment the MDA reaction is further incubated at a second temperature for about 10 min, preferably 10 min.

In one embodiment the second temperature is about 80° C., preferably at 80° C.

In one embodiment the MDA reaction comprises a buffer, deoxyribonucleotide triphosphates, dimethyl sulfoxide and T4Gene32 protein.

In one embodiment the MDA reaction contains less than 30 ng, preferably less than 15 ng, preferably less than 10 ng, preferably less than 5 ng, preferably less than 2 ng, preferably less than 0.2 ng, preferably less than 0.02 ng, preferably less than 0.002 ng of M. tuberculosis or M. bovis DNA.

In another aspect the invention relates to a method of selectively amplifying the genomic DNA of Mycobacterium tuberculosis from a sample, the method comprising:
contacting the sample with a composition comprising 7 to 12 unique oligonucleotide primers selected from the group consisting of P1-P14 and P15,
selectively amplifying DNA from M. tuberculosis in a multiple displacement amplification (MDA) reaction, and
identifying from among the selectively amplified DNA, DNA sequences that are assigned with high confidence to the genome of M. tuberculosis.

In one embodiment the composition comprises 7 to 15 unique oligonucleotide primers.

In one embodiment identifying comprises sequencing and bioinformatics analysis of the amplified DNA products.

In one embodiment the DNA sequences are identified as encoding bacterial gene products that are linked to or directly involved in conferring antibiotic resistance in M. tuberculosis.

In one embodiment the DNA sequences are identified as encoding proteins or portions thereof or RNAs or portions thereof that are linked to or directly involved in conferring antibiotic resistance in M. tuberculosis.

In one embodiment, identifying from among the selectively amplified DNA, DNA sequences that are assigned with high confidence to the genome of M. tuberculosis comprises whole genome sequencing (WGS) of the amplified DNA and bioinformatics analysis of the obtained nucleotide sequences to determine the nucleotide sequence of the M. tuberculosis genome.

In one embodiment, the method further comprises identifying from among the selectively amplified DNA, DNA sequences encoding bacterial gene products that are linked to or directly involved in conferring antibiotic resistance in this species or strain comprising generating an antibiotic resistance profile by whole genome sequencing (WGS) and bioinformatics analysis of the amplified DNA to determine the nucleotide sequence of at least one gene locus that is linked to or that is directly involved in antibiotic resistance in M. tuberculosis.

In one embodiment, generating the antibiotic resistance profile comprises WGS and bioinformatics analysis of the amplified DNA to determine the nucleotide sequence of at least one, preferably at least two, preferably at least 3, preferably at least 4, preferably at least 5, preferably at least 6, preferably at least 7, preferably at least 8, preferably at least 9, preferably at least 10, preferably at least 11, preferably at least 12, preferably 13 gene loci that are linked to or that are directly involved in antibiotic resistance in M. tuberculosis.

In one embodiment the gene loci are selected from the group consisting of alkyl hydroperoxidase reductase subunit C (ahpC), arabinosyl transferase B (embB), 7-methylguanosine methyltransferase (gidB), DNA gyrase (gyrA), DNA gyrase (gyrB), NADH-dependent enoyl-acyl carrier protein reductase (inhA), catalase/peroxidase (katG), pyrazinamidase/nicotinamidase (pncA), RNA polymerase β subunit (rpoB), ribosomal protein S12 (rpsL), 16S rRNA (rrs), thymidylate synthase (thyA) and rRNA methyltransferase (tlyA).

In some embodiments the method comprises identifying an antibiotic that is effective against M. tuberculosis based on the antibiotic resistance profile.

In one embodiment the sample is a culture of M. tuberculosis.

In one embodiment the culture is less than 5 days, preferably less than 4 days, preferably less than 3 days old.

In one embodiment the MDA reaction is carried out using a 029 polymerase.

In one embodiment the MDA reaction is carried out at a first temperature of about 25° C. to about 40° C., preferably about 26° C. to about 38° C., preferably about 27° C. to about 36° C., preferably about 28° C. to about 34° C., preferably about 29° C. to about 32° C., preferably about 30° C.

In one embodiment the MDA reaction is carried out at a first temperature of about 25° C. to about 45° C., preferably about 30° C. to about 45° C., preferably about 30° C. to about 44° C., preferably about 32° C. to about 43° C., preferably about 33° C. to about 42° C., preferably about 34° C. to about 41° C., preferably about 35° C. to about 40° C., preferably about 36° C. to about 39° C., preferably about 36° C. to about 38° C., preferably about 37° C.

In one embodiment the MDA reaction is carried out at a first temperature of 25° C. to 40° C., preferably 26° C. to 38° C., preferably 27° C. to 36° C., preferably 28° C. to 34° C., preferably 29° C. to 32° C., preferably 30° C.

In one embodiment the MDA reaction is carried out at a first temperature of 25° C. to 45° C., preferably 30° C. to 45° C., preferably 30° C. to 44° C., preferably 32° C. to 43° C., preferably 33° C. to 42° C., preferably 34° C. to 41° C., preferably 35° C. to 40° C., preferably 36° C. to 39° C., preferably 36° C. to 38° C., preferably 37° C.

In one embodiment the MDA reaction is incubated at a first temperature for at least 1 hour, preferably for at least 2 h, preferably for at least 3 h, preferably for at least 4 h, preferably for at least 5 h, preferably for at least 6 h, preferably for at least 7 h, preferably for at least 8 h, preferably for at least 9 h, preferably for at least 10 h, preferably for at least 11 h, preferably for at least 12 h, preferably for at least 13 h, preferably for at least 14 h, preferably for at least 15 h, preferably for at least 16 h.

In one embodiment the MDA reaction is carried out at a first temperature for up to 1 hour, preferably for up to 2 h, preferably for up to 3 h, preferably for up to 4 h, preferably for up to 5 h, preferably for up to 6 h, preferably for up to 7 h, preferably for up to 8 h, preferably for up to 9 h, preferably for up to 10 h, preferably for up to 11 h, preferably for up to 12 h, preferably for up to 13 h, preferably for up to 14 h, preferably for up to 15 h, preferably for up to 16 h.

In one embodiment the MDA reaction is carried out at a first temperature for about 1 hour, preferably for about 2 h, preferably for about 3 h, preferably for about 4 h, preferably for about 5 h, preferably for about 6 h, preferably for about 7 h, preferably for about 8 h, preferably for about 9 h, preferably for about 10 h, preferably for about 11 h, preferably for about 12 h, preferably for about 13 h, preferably for about 14 h, preferably for about 15 h, preferably for about 16 h.

In one embodiment the MDA reaction is carried out at a first temperature of about 25° C. to about 40° C., preferably about 26° C. to about 38° C., preferably about 27° C. to about 36° C., preferably about 28° C. to about 34° C., preferably about 29° C. to about 32° C., preferably about 30° C.

In one embodiment the MDA reaction is carried out at a first temperature of about 25° C. to about 45° C., preferably about 30° C. to about 45° C., preferably about 30° C. to about 44° C., preferably about 32° C. to about 43° C., preferably about 33° C. to about 42° C., preferably about 34° C. to about 41° C., preferably about 35° C. to about 40° C., preferably about 36° C. to about 39° C., preferably about 36° C. to about 38° C., preferably about 37° C.

In one embodiment the MDA reaction is carried out at a first temperature of 25° C. to 40° C., preferably 26° C. to 38° C., preferably 27° C. to 36° C., preferably 28° C. to 34° C., preferably 29° C. to 32° C., preferably 30° C.

In one embodiment the MDA reaction is carried out at a first temperature of 25° C. to 45° C., preferably 30° C. to 45° C., preferably 30° C. to 44° C., preferably 32° C. to 43° C., preferably 33° C. to 42° C., preferably 34° C. to 41° C., preferably 35° C. to 40° C., preferably 36° C. to 39° C., preferably 36° C. to 38° C., preferably 37° C.

In one embodiment the MDA reaction is further incubated at a second temperature for about 10 min, preferably for 10 min.

In one embodiment the second temperature is sufficient to inactivate the polymerase.

In one embodiment the second temperature is at about 65° C., preferably at 65° C.

In one embodiment the MDA reaction comprises a buffer, deoxyribonucleotide triphosphates, bovine serum albumin and trehalose-dihydrate. In one embodiment the MDA reaction comprises yeast inorganic pyrophosphatase and/or potassium chloride.

In one embodiment the MDA reaction contains less than 30 ng, preferably less than 15 ng, preferably less than 10 ng, preferably less than 5 ng, preferably less than 2 ng, preferably less than 0.2 ng, preferably less than 0.02 ng, preferably less than 0.002 ng of M. tuberculosis DNA.

In one embodiment the sample is a sample containing or suspected of containing DNA from M. tuberculosis and DNA from at least one other organism, preferably from at least 2, preferably from at least 5, preferably from at least 10, preferably from at least 15 other organisms.

In one embodiment the at least one other organism is selected from the group consisting of prokaryotes and eukaryotes. In one embodiment the prokaryotes are bacteria. In one embodiment the bacterial are Gram-negative or Gram-positive bacteria, or both.

In one embodiment the eukaryotes are protists or animals.

In one embodiment the animals are mammals.

In one embodiment the mammals are selected from the group consisting of humans, bovines, ovines, cervines, porcines, camelids, felines and canines.

In one embodiment the sample contains or also contains, DNA or RNA from a virus.

In one embodiment the sample is from a human.

In one embodiment the sample is from a cow.

In one embodiment the sample is a sputum sample.

In one embodiment the sample is a saliva sample.

In this embodiment the MDA reaction is carried out using a Bst polymerase.

In one embodiment the MDA reaction is carried out at a first temperature of 38° C. to 60° C., preferably 40° C. to 56° C., preferably 42° C. to 52° C., preferably 44° C. to about 48° C., preferably 45° C., preferably 46° C., preferably 47° C.

In one embodiment the MDA reaction is incubated at a first temperature for at least 1 hour, preferably for at least 2 h, preferably for at least 3 h, preferably for at least 4 h, preferably for at least 5 h, preferably for at least 6 h.

In one embodiment the MDA reaction is carried out at a first temperature for up to 1 hour, preferably for up to 2 h, preferably for up to 3 h, preferably for up to 4 h, preferably for up to 5 h, preferably for up to 6 h.

In one embodiment the MDA reaction is carried out at a first temperature for about 1 hour, preferably for about 2 h, preferably for about 3 h, preferably for about 4 h, preferably for about 5 h, preferably for about 6 h.

In one embodiment the MDA reaction is carried out at a first temperature for 1 hour, preferably for 2 h, preferably for 3 h, preferably for 4 h, preferably for 5 h, preferably for 6 h.

In one embodiment the MDA reaction is carried out at a first temperature of 38° C. to 60° C., preferably 40° C. to 56° C., preferably 42° C. to 52° C., preferably 44° C. to about 48° C., preferably 45° C., preferably 46° C., preferably 47° C.

In one embodiment the MDA reaction is further incubated at a second temperature for about 10 min, preferably for 10 min.

In one embodiment the second temperature is sufficient to inactivate the polymerase.

In one embodiment the second temperature is at about 80° C., preferably at 80° C.

In one embodiment the MDA reaction comprises a buffer, deoxyribonucleotide triphosphates, dimethyl sulfoxide and T4Gene32 protein.

In one embodiment the MDA reaction contains less than 30 ng, preferably less than 15 ng, preferably less than 10 ng, preferably less than 5 ng, preferably less than 2 ng, preferably less than 0.2 ng, preferably less than 0.02 ng, preferably less than 0.002 ng of M. tuberculosis DNA.

In addition, specifically contemplated as embodiments of this aspect of the invention that is a method of selectively amplifying the genomic DNA of M. tuberculosis are all of the embodiments of the invention set forth in the aspect of the invention that is a method of selectively amplifying the genomic DNA of at least one bacterial species or strain, including the unique oligonucleotide primers and combinations of unique oligonucleotide primers set forth in the composition aspects and embodiments of the invention and the use of such as set forth in the method and use aspects and embodiments of the invention.

In another aspect the invention relates to a method of selectively amplifying the genomic DNA of Mycobacterium bovis from a sample, the method comprising:
contacting the sample with a composition comprising 7 to 12 unique oligonucleotide primers selected from the group consisting of P1-P14 and P15,
selectively amplifying DNA from M. bovis in a multiple displacement amplification (MDA) reaction, and
identifying from among the selectively amplified DNA, DNA sequences that are assigned with high confidence to the genome of M. bovis.

In one embodiment the composition comprises 7 to 15 unique oligonucleotide primers.

Specifically contemplated as embodiments of this aspect of the invention that is a method of selectively amplifying the genomic DNA of M. bovis are all of the embodiments of the invention set forth in the aspect of the invention that is a method of selectively amplifying the genomic DNA of M. tuberculosis, including the unique oligonucleotide primers and combinations of unique oligonucleotide primers set forth in the composition aspects and embodiments of the invention and the use of such as set forth in the method and use aspects and embodiments of the invention.

In another aspect the invention relates to a method of determining the antibiotic resistance profile of a strain of M. tuberculosis, the method comprising:
contacting a sample containing or suspected of containing M. tuberculosis with a composition comprising 7 to 12 unique oligonucleotide primers selected from the group consisting of P1-P14 and P15,
selectively amplifying DNA from M. tuberculosis in a multiple displacement amplification (MDA) reaction, and
identifying within the pool of selectively amplified DNA, DNA sequences that encode M. tuberculosis gene products that are linked to, or that are directly involved in, antibiotic resistance in M. tuberculosis.

In one embodiment the composition comprises 7 to 15 unique oligonucleotide primers.

In one embodiment identifying comprises sequencing and bioinformatics analysis of the amplified DNA products.

In one embodiment the DNA sequences are identified as encoding bacterial gene products that are linked to or directly involved in conferring antibiotic resistance in M. tuberculosis.

In one embodiment the DNA sequences are identified as encoding proteins or portions thereof or RNAs or portions thereof that are linked to or directly involved in conferring antibiotic resistance in M. tuberculosis.

In one embodiment, identifying within the pool of selectively amplified DNA, DNA sequences that are assigned with high confidence to the genome of M. tuberculosis comprises whole genome sequencing (WGS) of the amplified DNA and bioinformatics analysis of the obtained nucleotide sequences to determine the nucleotide sequence of M. tuberculosis.

In one embodiment, identifying within the pool of selectively amplified DNA, DNA sequences encoding bacterial gene products that are linked to or directly involved in conferring antibiotic resistance in M. tuberculosis comprises generating an antibiotic resistance profile by whole genome sequencing (WGS) and bioinformatics analysis of the amplified DNA to determine the nucleotide sequence of at least one gene locus that is linked to or that is directly involved in antibiotic resistance in M. tuberculosis.

In one embodiment, generating the antibiotic resistance profile comprises WGS and bioinformatics analysis of the amplified DNA to determine the nucleotide sequence of at least one, preferably at least two, preferably at least 3, preferably at least 4, preferably at least 5, preferably at least 6, preferably at least 7, preferably at least 8, preferably at least 9, preferably at least 10, preferably at least 11, preferably at least 12, preferably 13 gene loci that are linked to or that are directly involved in antibiotic resistance in M. tuberculosis.

In one embodiment the gene loci are selected from the group consisting of alkyl hydroperoxidase reductase subunit C (ahpC), arabinosyl transferase B (embB), 7-methylguanosine methyltransferase (gidB), DNA gyrase (gyrA), DNA gyrase (gyrB), NADH-dependent enoyl-acyl carrier protein reductase (inhA), catalase/peroxidase (katG), pyrazinamidase/nicotinamidase (pncA), RNA polymerase β subunit (rpoB), ribosomal protein S12 (rpsL), 16S rRNA (rrs), thymidylate synthase (thyA) and rRNA methyltransferase (tlyA).

In some embodiments the method comprises identifying an antibiotic that is effective against M. tuberculosis based on the antibiotic resistance profile.

Specifically contemplated as embodiments of this aspect of the invention that is a method of determining the antibiotic resistance profile of a strain of M. tuberculosis are all of the embodiments of the invention set forth in the aspect of the invention that is a method of selectively amplifying the genomic DNA of M. tuberculosis, including the unique oligonucleotide primers and combinations of unique oligonucleotide primers set forth in the composition aspects and embodiments of the invention and the use of such in the method and use aspects and embodiments of the invention.

In another aspect the invention relates to a method of determining the antibiotic resistance profile of a strain of M. bovis, the method comprising:
contacting a sample containing or suspected of containing M. tuberculosis with a composition comprising 7 to 12 unique oligonucleotide primers selected from the group consisting of P1-14 and P15,
selectively amplifying DNA from M. bovis in a multiple displacement amplification (MDA) reaction, and
identifying within the pool of selectively amplified DNA, DNA sequences that encode M. bovis gene products that are linked to, or that are directly involved in, antibiotic resistance in M. bovis.

In one embodiment the composition comprises 7 to 15 unique oligonucleotide primers.

In one embodiment identifying comprises sequencing and bioinformatics analysis of the amplified DNA products.

In one embodiment the DNA sequences are identified as encoding bacterial gene products that are linked to or directly involved in conferring antibiotic resistance in M. bovis.

In one embodiment the DNA sequences are identified as encoding proteins or portions thereof or RNAs or portions thereof that are linked to or directly involved in conferring antibiotic resistance in M. bovis.

In one embodiment, identifying from among the selectively amplified DNA, DNA sequences that are assigned with high confidence to the genome of M. bovis comprises whole genome sequencing (WGS) of the amplified DNA and bioinformatics analysis of the obtained nucleotide sequences to determine the nucleotide sequence of M. bovis.

In one embodiment, identifying from among the selectively amplified DNA, DNA sequences encoding bacterial gene products that are linked to or directly involved in conferring antibiotic resistance in M. bovis comprises generating an antibiotic resistance profile by whole genome sequencing (WGS) and bioinformatics analysis of the amplified DNA to determine the nucleotide sequence of at least one gene locus that is linked to or that is directly involved in antibiotic resistance in M. bovis.

In one embodiment, the method further comprises identifying an antibiotic that is or is expected to be effective against M. bovis based on the antibiotic resistance profile.

In one embodiment, generating the antibiotic resistance profile comprises WGS and bioinformatics analysis of the amplified DNA to determine the nucleotide sequence of at least one, preferably at least two, preferably at least 3, preferably at least 4, preferably at least 5, preferably at least 6, preferably at least 7, preferably at least 8, preferably at least 9, preferably at least 10, preferably at least 11, preferably at least 12, preferably 13 gene loci that are linked to or that are directly involved in antibiotic resistance in M. bovis.

In one embodiment the gene loci are selected from the group consisting of alkyl hydroperoxidase reductase subunit C (ahpC), arabinosyl transferase B (embB), 7-methylguanosine methyltransferase (gidB), DNA gyrase (gyrA), DNA gyrase (gyrB), NADH-dependent enoyl-acyl carrier protein reductase (inhA), catalase/peroxidase (katG), pyrazinamidase/nicotinamidase (pncA), RNA polymerase β subunit (rpoB), ribosomal protein S12 (rpsL), 16S rRNA (rrs), thymidylate synthase (thyA) and rRNA methyltransferase (tlyA).

Specifically contemplated as embodiments of this aspect of the invention that is a method of determining the antibiotic resistance profile of a strain of M. bovis are all of the embodiments of the invention set forth in the aspect of the invention that is a method of selectively amplifying the genomic DNA of M. tuberculosis, including the unique oligonucleotide primers and combinations of unique oligonucleotide primers set forth in the composition aspects and embodiments of the invention and the use of such in the method and use aspect and embodiments of the invention.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents; or such sources of information, in any jurisdiction, are prior art, or form part of the common general knowledge in the art.

The invention will now be illustrated in a non-limiting way by reference to the following examples.

### EXAMPLES

### Materials and Methods

### MDA Primer Selection

A subset of 12 MDA primers was selected from the full set of 15. Eleven of the 12 MDA primers were selected on the basis that for these eleven primers the possible binding positions on the M. tuberculosis genome included at least one that was within 5 kb, in either the upstream or downstream directions, of one or more of 13 genes commonly linked to or directly involved in antibiotic resistance in M. tuberculosis (Table 2). The twelfth primer was selected primarily because it binds frequently to the M. tuberculosis genome. The inclusion of this primer also ensured that at least one binding position is within 10 kb, in both the upstream or downstream directions, for all 13 genes commonly linked to or directly involved in antibiotic resistance in M. tuberculosis (Table 2).

### Genomic DNA

A sample of M. tuberculosis DNA was provided by collaborators at Otago University. This sample (denoted 5734) was obtained from a M. tuberculosis strain that was cultured on Lowenstein-Jensen media for 6-8 weeks and the DNA extracted using the UltraClean® Microbial DNA isolation and purification kit (Qiagen). Following purification the DNA sample was incubated in a boiling water bath for 10 minutes to ensure no viable bacteria remained.

MDA of M. tuberculosis DNA

To provide template for WGS analyses the M. tuberculosis DNA was enriched using the 12 selected MDA primers (SEQ ID NO: 1-SEQ ID NO: 12) with other reaction conditions following the manufacturers recommendations for 029 polymerase, reaction buffer, DTT, and bovine serum albumin (New England BioLabs, USA). Exceptions were that the reaction volume was halved to 25 ul although the quantity of primer (125 pmol of each) and template (10-20 ng) added was as recommended for a 50 ul reaction volume. Amplification reactions were incubated in a thermocycler at 30° C. for 6 or 16 hours followed by 15 minutes at 85° C.

Following incubation the reaction products were used directly for Illumina and Oxford Nanopore sequencing library preparation.

### Illumina Short Read Sequencing

Products from targeted MDA reaction were prepared for WGS using the Illumina Nextera XT DNA library preparation kit following recommended protocols (e.g. Lamble et al. 2013; Tyler et al. 2016). Briefly, the input DNA was enzymatically cleaved into fragments approximately 300 bp long and then tagged with specific adapters. After adapter ligation, indexes were added using a 15-cycle PCR amplification.

High throughput sequencing was performed on Illumina MiSeq instruments using Illumina MiSeq Reagent kit v2 (300-cycle) to generate 150 nucleotide paired-end sequence reads. Raw sequence reads were processed using a standard workflow in Trimmomatic v0.35 (Bolger et al. 2014); this removes sequences corresponding to the Illumina adapters as well as regions of low quality (i.e., phred score<33). Processed sequence reads were then mapped against the M. tuberculosis H37rv reference genome using BWA 0.7 (Li & Durbin 2009) and sequence positions known to be associated with antibiotic resistance evaluated to determine a drug resistance profile for the strain.

### Oxford Nanopore MinION Sequencing

Products from targeted MDA reaction were prepared for WGS using the Oxford Nanopore 1D² ligation sequencing kit following the manufacturer recommended protocol. Briefly, the input DNA is blunt-ended repaired before having flow cell adapters and a hairpin linker (for reverse complementary reads) added. To obtain the final library, DNA fragments were purified using a standard magnetic bead approach.

After QC of the MinION flow cell (FLO-MIN 106 R9.4), the DNA library was loaded and a standard 48 hour sequencing run initiated using the MinKNOW ONT software. Following run completion, sequence reads were mapped against the M. tuberculosis H37rv reference genome using Geneious 9.0 (Kearse et al. 2012) and sequence positions known to be associated with antibiotic resistance evaluated to determine a drug resistance profile for the strain.

### Results

The results of the above design and validation efforts are presented in the following tables (Tables 2-5).

TABLE 2
Number of binding sites for the 15 M. tuberculosis MDA oligonucleotide primers on the M. tuberculosis H37rv
reference genome and on the genomes of 15 other bacteria commonly found in the upper respiratory tract of humans.

| | Taxa^{a} | | | | | | | | |
|---|---|---|---|---|---|---|---|---|---|
| Primer | Mycobacterium | Haemophilus | Chlamydophila | Pseudomonas | Escherichia | Bordetella | Neisseria | Listeria | Lactobacillus |
| name | tuberculosis | influenzae | pneumoniae | aeruginosa | coli | pertussis | meningitidis | monocytogenes | brevis |
| P1 | 4 | 0 | 0 | 0 | 1 | 2 | 0 | 0 | 1 |
| P2 | 21 | 0 | 0 | 4 | 2 | 1 | 1 | 0 | 0 |
| P3 | 17 | 0 | 0 | 5 | 0 | 1 | 1 | 0 | 1 |
| P4 | 6 | 1 | 0 | 2 | 0 | 0 | 0 | 0 | 0 |
| P5 | 21 | 0 | 0 | 4 | 4 | 4 | 0 | 0 | 0 |
| P6 | 15 | 0 | 0 | 3 | 0 | 0 | 5 | 0 | 0 |
| P7 | 6 | 0 | 0 | 1 | 0 | 2 | 0 | 0 | 0 |
| P8 | 6 | 0 | 0 | 1 | 0 | 4 | 0 | 0 | 0 |
| P9 | 3 | 0 | 0 | 0 | 0 | 0 | 0 | 0 | 0 |
| P10 | 7 | 0 | 0 | 1 | 0 | 1 | 0 | 0 | 0 |
| P11 | 10 | 0 | 0 | 2 | 0 | 0 | 0 | 0 | 0 |
| P12 | 179 | 0 | 0 | 47 | 2 | 30 | 12 | 0 | 1 |
| P13 | 87 | 0 | 0 | 7 | 1 | 30 | 0 | 0 | 0 |
| P14 | 567 | 1 | 0 | 42 | 27 | 16 | 48 | 1 | 4 |
| P15 | 475 | 2 | 0 | 43 | 25 | 24 | 46 | 0 | 5 |

| | Taxa^{a} | | | | | | |
|---|---|---|---|---|---|---|---|
| Primer | Leuconostoc | Clostridioides | Porphyromonas | Veillonella | Moraxella | Enterobacter | Staphylococcus |
| name | mesenteroides | difficile | gingivalis | parvula | catarrhalis | aerogenes | aureus |
| P1 | 0 | 0 | 0 | 1 | 0 | 3 | 0 |
| P2 | 0 | 0 | 0 | 0 | 0 | 0 | 0 |
| P3 | 0 | 0 | 1 | 0 | 0 | 1 | 0 |
| P4 | 0 | 0 | 0 | 0 | 0 | 0 | 0 |
| P5 | 0 | 0 | 0 | 0 | 0 | 0 | 0 |
| P6 | 0 | 0 | 0 | 0 | 0 | 2 | 0 |
| P7 | 0 | 0 | 1 | 0 | 0 | 1 | 0 |
| P8 | 0 | 0 | 0 | 0 | 0 | 2 | 0 |
| P9 | 0 | 0 | 0 | 0 | 0 | 0 | 0 |
| P10 | 1 | 0 | 0 | 0 | 0 | 2 | 0 |
| P11 | 0 | 0 | 0 | 0 | 1 | 0 | 0 |
| P12 | 0 | 0 | 0 | 0 | 0 | 24 | 0 |
| P13 | 0 | 0 | 1 | 0 | 0 | 1 | 0 |
| P14 | 0 | 0 | 0 | 0 | 3 | 28 | 0 |
| P15 | 1 | 0 | 1 | 0 | 2 | 25 | 0 |

^{a}GenBank accession numbers for genomes included in comparison. Mycobacterium tuberculosis (NC_000962), Haemophilus influenzae (NC_000907), Chlamydophila pneumoniae (NC_000922), Pseudomonas aeruginosa (NC_002516), Escherichia coli (NC_002695), Bordetella pertussis (NC_002929), Neisseria meningitidis (NC_003112), Listeria monocytogenes (NC _{—}003210), Lactobacillus brevis (NC_008497), Leuconostoc mesenteroides (NC_008531), Clostridioides difficile (NC_009089), Porphyromonas gingivalis (NC_010729), Veillonellaparvula (NC_013520), Moraxella catarrhalis (NC_014147), Enterobacter aerogenes (NC_015663), Staphylococcus aureus (NZ_CP010295).

TABLE 3
Positions, when mapped to the M. tuberculosis H37rv reference genome, of the 15 MDA oligonucleotide
primers relative to those of the 13 recognised antibiotic resistance loci.

| | Closet 5′ | | Other primers within | Closet 3′ | | Other primers within |
|---|---|---|---|---|---|---|
| Gene | MDA | Distance, in | 15 kb of the | MDA | Distance, in | 15 kb of the |
| locus | primer^{a} | nucleotides | gene target^{a} | primer^{a} | nucleotides | gene target^{a} |
| ahpC | P1 | 4020 | — | P11 | 4266 | — |
| embB | P3 | 1532 | — | P8 | 3481 | P3 |
| gid | P10 | 1615 | — | P6 | 2366 | — |
| gyrA | P2 | 2592 | P10 | P12 | 5217 | — |
| gyrB | P2 | 530 | P10 | P12 | 7768 | — |
| inhA | P10 | 2517 | — | P4 | 1229 | P6 |
| katG | P1 | 288 | — | P11 | 3182 | — |
| pncA | P7 | 1086 | — | P9 | 3866 | P5 |
| rpoB | P8 | 4628 | P13 | P4 | 1525 | P9, P12, P13, P15 |
| rpsL | P2 | 613 | P13 | P5 | 5587 | — |
| rrs | P8 | 2639 | — | P5 | 5137 | P12, P13 |
| thyA | P7 | 826 | P12, P13 | P6 | 841 | — |
| tlyA | P3 | 2329 | P5 | P12 | 4762 | — |

TABLE 4
Genome coverage statistics and antibiotic resistance profile calls for the original non-amplified sample 5734 and targeted MDA reactions
of 6 hr and 16 hr using 10-20 ng DNA as starting template when mapped to the M. tuberculosis H37rv reference genome.

| | Percentage | | | Percentage | | |
|---|---|---|---|---|---|---|
| | of reads | | Range of | of reference | Drug resistance loci with | |
| | mapping to | Mean depth | coverage | genome | recognized resistance | Inferred drug |
| Sample | reference | of coverage | depths | covered | inducing mutation | resistance profile |
| non-amplified | 99.1 | 23.4 | 0-415 | 99.0 | gyrA, rpoB, rpSL, rrs, katG, embB | FQ, RMP, SM, AMK, KAN, CPR, |
| | | | | | | INH, EMB |
| 6 hr MDA Illumina | 99.3 | 20.2 | 0-223 | 99.2 | gyrA, rpoB, rpSL, rrs, katG, embB | FQ, RMP, SM, AMK, KAN, CPR, |
| | | | | | | INH, EMB |
| 16 hr MDA Illumina | 99.4 | 18.8 | 0-219 | 99.5 | gyrA, rpoB, rpSL, rrs, katG, embB | FQ, RMP, SM, AMK, KAN, CPR, |
| | | | | | | INH, EMB |
| 16 h MDA MinION | 99.5 | 19.2 | 0-224 | 100.0 | gyrA, rpoB, rpSL, rrs, katG, embB | FQ, RMP, SM, AMK, KAN, CPR, |
| | | | | | | INH, EMB |

TABLE 5
Gene-by-gene coverage statistics for the original non-amplified
sample 5734 and targeted MDA reactions of 6 hr and 16 hr
using 10-20 ng DNA as starting template when mapped to the
M. tuberculosis H37rv reference genome.

| | | 6 hr | 16 hr | 16 h |
|---|---|---|---|---|
| | non- | MDA | MDA | MDA |
| Gene locus | amplified | Illumina | Illumina | MinION |
| ahpC | | | | |
| Mean depth of coverage | 14.6 | 26.9 | 28.2 | 29.1 |
| Range of coverage depths | 7-23 | 18-36 | 21-38 | 21-40 |
| Percentage of gene | 100% | 100% | 100% | 100% |
| reference covered | | | | |
| embB | | | | |
| Mean depth of coverage | 18.8 | 19.6 | 17.8 | 17.7 |
| Range of coverage depths | 7-34 | 9-36 | 8-29 | 8-29 |
| Percentage of gene | 100% | 100% | 100% | 100% |
| reference covered | | | | |
| gid | | | | |
| Mean depth of coverage | 27.1 | 36.5 | 29.3 | 28.7 |
| Range of coverage depths | 18-36 | 29-45 | 22-36 | 21-37 |
| Percentage of gene | 100% | 100% | 100% | 100% |
| reference covered | | | | |
| gyrA | | | | |
| Mean depth of coverage | 27.5 | 26.0 | 24.5 | 24.1 |
| Range of coverage depths | 15-42 | 15-51 | 12-40 | 11-41 |
| Percentage of gene | 100% | 100% | 100% | 100% |
| reference covered | | | | |
| gyrB | | | | |
| Mean depth of coverage | 23.9 | 31.5 | 28.9 | 28.6 |
| Range of coverage depths | 11-39 | 21-51 | 18-42 | 18-41 |
| Percentage of gene | 100% | 100% | 100% | 100% |
| reference covered | | | | |
| inhA | | | | |
| Mean depth of coverage | 29 | 18.0 | 17.6 | 17.1 |
| Range of coverage depths | 15-51 | 12-24 | 11-24 | 11-26 |
| Percentage of gene | 100% | 100% | 100% | 100% |
| reference covered | | | | |
| katG | | | | |
| Mean depth of coverage | 24.2 | 18.4 | 17.8 | 17.6 |
| Range of coverage depths | 0-415 | 14-27 | 8-32 | 7-31 |
| Percentage of gene | 99% | 100% | 100% | 100% |
| reference covered | | | | |
| pncA | | | | |
| Mean depth of coverage | 20.4 | 18.2 | 18.4 | 17.8 |
| Range of coverage depths | 14-28 | 10-29 | 13-28 | 12-26 |
| Percentage of gene | 100% | 100% | 100% | 100% |
| reference covered | | | | |
| rpoB | | | | |
| Mean depth of coverage | 22.1 | 22.8 | 21.7 | 21.3 |
| Range of coverage depths | 12-33 | 11-41 | 12-37 | 11-36 |
| Percentage of gene | 100% | 100% | 100% | 100% |
| reference covered | | | | |
| rpsL | | | | |
| Mean depth of coverage | 32.6 | 23.9 | 24.4 | 24.5 |
| Range of coverage depths | 26-40 | 14-37 | 17-32 | 19-33 |
| Percentage of gene | 100% | 100% | 100% | 100% |
| reference covered | | | | |
| rrs | | | | |
| Mean depth of coverage | 42.8 | 37.6 | 35.4 | 34.6 |
| Range of coverage depths | 16-74 | 24-51 | 23-47 | 18-46 |
| Percentage of gene | 100% | 100% | 100% | 100% |
| reference covered | | | | |
| thyA | | | | |
| Mean depth of coverage | 17.9 | 21.6 | 19.4 | 19.2 |
| Range of coverage depths | 9-24 | 11-30 | 12-25 | 11-26 |
| Percentage of gene | 100% | 100% | 100% | 100% |
| reference covered | | | | |
| tlyA | | | | |
| Mean depth of coverage | 18.8 | 21.8 | 16.6 | 12.1 |
| Range of coverage depths | 11-30 | 14-28 | 21-38 | 8-28 |
| Percentage of gene | 100% | 100% | 100% | 100% |
| reference covered | | | | |

### Discussion

As provided in Tables 1-5 above and the drawings, the inventors have developed a set of oligonucleotide primers that when used with either the 029 or Bst enzymes under standard MDA conditions, result in the selective amplification of M. tuberculosis genomic DNA. This has potential application when genotyping M. tuberculosis from small amounts of starting material and for WGS (Illumina and MinION) sequencing of M. tuberculosis genomes from young cultures and sputum samples.

Sequencing of these templates on Illumina MiSEQ and Oxford Nanopore MinION instruments resulted in percentage of mapped sequence reads and depth of coverage statistics highly similar to those for the non-MDA control. Specifically, for the non-MDA control 99.1% of reads mapped to the reference genome with an average genome coverage of 23.4 times and 17.3-42.5 times for each of the gene loci currently associated with antibiotic resistance. For MDA products the percentage of mapped sequence reads were 99.3-99.5% and when the number of reads collected were standardised, coverage of 18.8-20.2 and 16.1-37.2 for the genome as a whole and individual genes, respectively.

While overall coverage was marginally lower for templates produced by MDA, for several gene loci the depth of coverage was greater for these templates. For example, the average coverage of the gid locus was 24.2 for the non-MDA control but 28.7-36.0 for the MDA produced templates. Importantly, templates produced by MDA provided the same antibiotic resistance profile as the non-MDA control.

Existing approaches for evaluating the drug susceptibility of M. tuberculosis isolated from patients have several limitations including requirements for specialised infrastructure, slow diagnosis and incomplete characterisation of antibiotic resistance. However, the effective implementation of the unique oligonucleotide primers and methods of selective amplification of the invention as described herein provides an advantage by allowing the rapid establishment of complete antibiotic resistance profiles for individual TB patients.

Whole genome sequencing has become the first choice for diagnosis of MDR-TB and XDR-TB as it allows genome wide assessment of genetic mutation and, thereby, a complete characterisation of antibiotic resistance. However, this activity is typically limited to well-resourced, centralised research laboratories because of the infrastructure requirements (e.g., large, expensive instruments that require controlled conditions and specialised maintenance). By employing the selective MDA primers and methods as described herein, WGS can also be effectively deployed to support clinical diagnosis of drug susceptibility for M. tuberculosis isolated from patients. Specifically, using these methods it would no longer be necessary to isolate and culture M. tuberculosis from a sputum sample prior to WGS in a centralised laboratory, reducing the time to diagnosis by up to several weeks.

The MDA-based methods described herein also enable WGS-based diagnosis to be performed in low infrastructure, point of care settings. By employing the MDA-based methods described herein sufficient quantities of DNA can be produced to allow WGS using the Oxford Nanopore MinION platform. This personal DNA sequencing device has few infrastructure requirements and when used to analyse DNA templates produced using selective MDA could enable rapid—on the order of hours not weeks—TB diagnosis at point of care.

### REFERENCES

Bolger, A. M. et al. (2014) Trimmomatic: A flexible trimmer for Illumina sequence data. Bioinformatics 30: 2114-2120.
Brown, A. C. et al. (2015). Rapid Whole-Genome sequencing of Mycobacterium tuberculosis isolates directly from clinical samples. Journal of Clinical Microbiology 53: 2230-2237.
Callaway, E. (2017). TB diagnostic test fails to curb cases. Nature 551: 424-425.
Clarke, E. L. et al. (2017) Swga: A Primer Design Toolkit for Selective Whole Genome Amplification. Bioinformatics 33: 2071-2077.
Colman, R. E. et al. (2016) Rapid drug susceptibility testing of drug resistant Mycobacterium tuberculosis directly from clinical samples using amplicon sequencing: a proof of concept study. Journal of Clinical Microbiology 54: 2058-2067.
Doherty, R. (2014) In the News: Antibiotic Resistance. British Dental Journal 216: 56.
Dookie, N. et al. (2018) Evolution of drug resistance in Mycobacterium tuberculosis: a review on the molecular determinants of resistance and implications for personalized care. Journal of Antimicrobial Chemotherapy 73: 1138-1151.
Evans, C. (2011) GeneXpert—a game-changer for tuberculosis control? PLOS Medicine 8: e1001064.
Feuerriegel, S. et al. (2015) PhyResSE: a web tool delineating Mycobacterium tuberculosis antibiotic resistance and lineage from whole-genome sequencing data. Journal of Clinical Microbiology 53: 1908-1914.
Gadkar, V. J. et al. (2014) Fidelity and representativeness of two isothermal multiple displacement amplification systems to preamplify limiting amounts of total RNA. Moecularl Biotechnlology 56: 377-385.
Gilpin, C. et al. (2016) Current tools available for the diagnosis of drug-resistant tuberculosis. Therapeutic Advances in Infectious Disease 3: 145-151.
Houben, R. M. G. J. & Dodd, P. J. (2016) The global burden of latent tuberculosis infection: a re-estimation using mathematical modelling. PLOS Medicine 13: e1002152.
Kane, Y. et al. (2016) Relevance of the GeneXpert test for the diagnosis of TB in chronic hemodialysis patients in Casamance, South of Senegal. Journal of Kidney 2: 1000133.
Kearse, M. et al. (2012) Geneious Basic: an integrated and extendable desktop software platform for the organization and analysis of sequence data. Bioinformatics 28: 1647-1649.
Klopper, M. et al. (2013) Emergence and spread of extensively and totally drug-resistant tuberculosis, South Africa. Emerging Infectious Diseases 19: 449-455.
Lamble, S. (2013). Improved workflows for high throughput library preparation using the transposome-based nextera system. BMC Biotechnology 13: 104.
Leichty, A. R. & Brisson, B. (2014) Selective whole genome amplification for resequencing target microbial species from complex natural samples. Genetics 198: 473-481.
Li, H. & Durbin, R. (2009) Fast and accurate short read alignment with Burrows-Wheeler Transform. Bioinformatics 25: 1754-1760.
Migliori, G. B. et al. (2008) Diagnosis of multidrug-resistant tuberculosis and extensively drug-resistant tuberculosis: current standards and challenges. The Canadian Journal of Infectious diseases and Medical Microbiology 19: 169-172.
Mortimer, T. D. et al. (2018) Signatures of selection at drug resistance loci in Mycobacterium tuberculosis. mSystems 3: e00108-17.
Nguyen, L. (2017) Antibiotic resistance mechanisms in M. tuberculosis: an update. Archives of Toxicology 90: 1585-1604.
Pan, X. et al. 2008. A Procedure for highly specific, sensitive, and unbiased whole-genome amplification. Proceedings of the National Academy of Sciences 105: 15499-15504.
Rufai, S. B et al. (2014) Comparison of Xpert MTB/RIF with line probe assay for detection of Rifampin-monoresistant Mycobacterium tuberculosis. Journal of Clinical Microbiology 52: 1846-1852.
Tyler, A. D. et al. (2016). Comparison of sample preparation methods used for the next-generation sequencing of Mycobacterium tuberculosis. PLoS One, 11(2), e0148676.
Zeka, A. N. et al. (2011) Evaluation of the GeneXpert MTB/RIF assay for rapid diagnosis of tuberculosis and detection of Rifampin resistance in pulmonary and extrapulmonary specimens. Journal of Clinical Microbiology 49: 4138-4141.

### INDUSTRIAL APPLICATION

The oligonucleotide primers and methods of using such according to the invention have industrial application in molecular biology in providing a rapid way to identify pathogenic strains of bacteria, particularly Mycobacterium tuberculosis and M. bovis.

1. A composition comprising 7 to 12 unique oligonucleotide primers, each primer consisting of 11 or 12 nucleotides, wherein each of these oligonucleotide primers specifically binds to a nucleic acid sequence in the M. tuberculosis genome.

2. The composition of claim 1 comprising 7 to 15 unique oligonucleotide primers.

3. The composition of claim 1 comprising at least 7 unique oligonucleotide primers selected from the group consisting of P1 (SEQ ID NO: 1), P2 (SEQ ID NO: 2), P3 (SEQ ID NO: 3), P4 (SEQ ID NO: 4), P5 (SEQ ID NO: 5), P6 (SEQ ID NO: 6), P7 (SEQ ID NO: 7), P8 (SEQ ID NO: 8), P9 (SEQ ID NO: 9), P10 (SEQ ID NO: 10), P11 (SEQ ID NO: 11), P12 (SEQ ID NO: 12), P13 (SEQ ID NO: 13), P14 (SEQ ID NO: 14) and P15 (SEQ ID NO: 15).

4. The composition of claim 3, wherein the oligonucleotide primers comprise P1-P6 and P12.

5. The composition of claim 3, wherein the oligonucleotide primers consist essentially of P1-P6 and P12.

6. The composition of claim 1, further comprising at least one enzyme that catalyses nucleic acid amplification.

7. The composition of claim 1, further comprising a Φ29 polymerase or a Bst polymersase.

8. A method of selectively amplifying the genomic DNA of at least one bacterial species or strain from a sample, the method comprising:
contacting the sample with a composition comprising 7-12 unique oligonucleotide primers, each primer consisting of 11 or 12 nucleotides, wherein each of these oligonucleotide primers specifically binds to a nucleic acid sequence in the genome of the bacterial species or strain,
selectively amplifying DNA from the bacterial species or strain of interest in a multiple displacement amplification (MDA) reaction,
identifying from among the selectively amplified DNA, DNA sequences that are assigned with high confidence to the genome of the at least one bacterial species or strain.

9. The method of claim 8, wherein the unique oligonucleotide primers are as defined in the composition of claim 3.

10. The method of claim 8, wherein the bacterial species or strain is a Mycobacterium spp.

11. The method of claim 8, wherein the bacterial species or strain is M. tuberculosis or M. bovis.

12. The method of claim 8, wherein the sample is a sample containing or suspected of containing DNA from M. tuberculosis or M. bovis, and DNA from at least one other organism.

13. The method of claim 8, wherein the sample is a sputum or saliva sample.

14. The method of claim 8, wherein the sample is from a human or from a bovine.

15. A method of determining the antibiotic resistance profile of a strain of Mycobacterium, the method comprising:
contacting a sample containing or suspected of containing at least one Mycobacterium spp. with a composition comprising 7 to 12 unique oligonucleotide primers selected from the group consisting of P1-P14 and P15,
selectively amplifying DNA from the at least one Mycobacterium spp. in a multiple displacement amplification (MDA) reaction, and
identifying within the pool of selectively amplified DNA, DNA sequences that encode at least one Mycobacterium spp. gene product that is linked to, or that is directly involved in, antibiotic resistance in the at least one Mycobacterium spp.

16. The method of claim 15, wherein identifying within the pool of selectively amplified DNA, DNA sequences encoding bacterial gene products that are linked to or directly involved in conferring antibiotic resistance in at least one Mycobacterium spp. comprises generating an antibiotic resistance profile by whole genome sequencing (WGS) and bioinformatics analysis of the amplified DNA to determine the nucleotide sequence of at least one gene locus that is linked to or that is directly involved in antibiotic resistance in at least one Mycobacterium spp.

17. The method of claim 16 wherein the gene loci are selected from the group consisting of alkyl hydroperoxidase reductase subunit C (ahpC), arabinosyl transferase B (embB), 7-methylguanosine methyltransferase (gidB), DNA gyrase (gyrA), DNA gyrase (gyrB), NADH-dependent enoyl-acyl carrier protein reductase (inhA), catalase/peroxidase (katG), pyrazinamidase/nicotinamidase (pncA), RNA polymerase β subunit (rpoB), ribosomal protein S12 (rpsL), 16S rRNA (rrs), thymidylate synthase (thyA) and rRNA methyltransferase (tlyA).

18. The method of claim 15, wherein the unique oligonucleotide primers are as defined in the composition of claim 3.

19. The method of claim 15, wherein the Mycobacterium spp. is M. tuberculosis or M. bovis.


SEQUENCE LISTING (15 sequences)

SEQ ID NO: 1 - DNA, 12 bp, Artificial Sequence
  Primer
aatggccgtcgc

SEQ ID NO: 2 - DNA, 12 bp, Artificial Sequence
  Primer
ggtcggtgcggg

SEQ ID NO: 3 - DNA, 12 bp, Artificial Sequence
  Primer
tggccggggtgt

SEQ ID NO: 4 - DNA, 12 bp, Artificial Sequence
  Primer
gcaacaccgggt

SEQ ID NO: 5 - DNA, 12 bp, Artificial Sequence
  Primer
gcgggcacggtg

SEQ ID NO: 6 - DNA, 12 bp, Artificial Sequence
  Primer
cgtcggctgcgg

SEQ ID NO: 7 - DNA, 12 bp, Artificial Sequence
  Primer
ccacccgcgcaa

SEQ ID NO: 8 - DNA, 12 bp, Artificial Sequence
  Primer
gacgcgcccacg

SEQ ID NO: 9 - DNA, 12 bp, Artificial Sequence
  Primer
tcgctacccacg

SEQ ID NO: 10 - DNA, 12 bp, Artificial Sequence
  Primer
atgttggtgatc

SEQ ID NO: 11 - DNA, 12 bp, Artificial Sequence
  Primer
ggtgtcgacgag

SEQ ID NO: 12 - DNA, 12 bp, Artificial Sequence
  Primer
cggcgacggcgg

SEQ ID NO: 13 - DNA, 12 bp, Artificial Sequence
  Primer
tgcgtctgctcg

SEQ ID NO: 14 - DNA, 11 bp, Artificial Sequence
  Primer
ccgccgttgcc

SEQ ID NO: 15 - DNA, 11 bp, Artificial Sequence
  Primer
ccgttgccgcc